(19) United States
(12) Patent Application Publication
(10) Pub. No.: US 2024/0299769 A1
(43) Pub. Date: Sep. 12, 2024

(54) BIOCERAMIC COMPOSITIONS FOR CANCER RECOVERY

(71) Applicant: MULTIPLE ENERGY TECHNOLOGIES LLC, Washington, PA (US)

(72) Inventors: Shannon VISSMAN, Washington, PA (US); Melody VISSMAN, Washington, PA (US); Francisco Jose CIDRAL-FILHO, Washington, PA (US)

(21) Appl. No.: 18/550,428

(22) PCT Filed: Mar. 15, 2022
(86) PCT No.: PCT/US2022/020445

§ 371 Date: Sep. 13, 2023

Related U.S. Application Data
(60) Provisional application No. 63/161,775, filed on Mar. 16, 2021.

(51) Int. Cl. A61N 5/06

(52) U.S. Cl. CPC A61N 5/0618; A61N 2005/0645

(57) ABSTRACT

The subject matter described herein is directed to articles, compositions, systems, and methods of using bioceramic compositions for the treatment and alleviation of side effects of cancer therapies, as well as enhancing the recovery of patients receiving cancer therapies.

### CROSS-REFERENCE

This application is a U.S. National Stage of International Application No. PCT/US2022/020445, filed on Mar. 15, 2022, which claims the benefit of priority to U.S. Provisional Patent Application No. 63/161,775, filed on Mar. 16, 2021, each of which are entirely incorporated herein by reference.

### BACKGROUND OF THE INVENTION

Infrared wavelength ranges from 0.7 to 1000 microns and is just beyond visible light on the electromagnetic spectrum. Infrared has strong physical properties and great thermal activity.

### SUMMARY OF THE INVENTION

The natural resonant frequency range of water and living organisms, including man, falls within the infrared range. For example, the wavelength range of 6-18 micrometers is beneficial to the human body by virtue of its activating and energizing effect on the body. Indeed, human skin radiates 9.36 micrometer infrared wave which is very close to the resonant frequency of a water molecule—and rightly so since our bodies are about 70% water. Infrared waves are considered a safe and beneficial energy source for humans. The instant inventors have identified beneficial properties of the inventive bioceramic compositions and applications as described herein.

As described herein, bioceramics include ceramics which radiate beneficial infrared waves to living organisms. The subject matter described herein utilizes the beneficial effects of the infrared radiation. The methods, articles, systems, and compositions of matter described herein employ a unique formulation of bioceramic materials, which are ultra-fine mineral particles, that when heated by a living organism, such as the human body, emit far-infrared energy. The bioceramic materials described herein are refractory polycrystalline compounds that due to their inertness in aqueous conditions are highly biocompatible and safe for human interaction and application. The inventors have invented numerous biomodulatory or physiological applications of these bioceramic formulations, including but not limited to the regulation of cell metabolism, the induction of analgesia, muscle relaxation and modulation of inflammation and oxidative stress.

According to the laws of thermodynamics, any two bodies in contact reach thermal equilibrium through a direct microscopic exchange of kinetic energy in the form of electromagnetic radiation generated by the thermal motion of the charged particles in matter. Thus, when the bioceramic materials, articles, and compositions described herein and the human body are in contact, there is an exchange of thermal radiation, more specifically far infrared radiation. Because of the specific properties of the minerals and oxides contained in the subject matter described herein, i.e., highly refractory minerals, this emission is intensified in the spectrum of far infrared which has numerous biomodulatory or physiological effects. The inventors of the instant application have unexpectedly discovered numerous advantages of using the bioceramic materials described herein to complement or serve as the basis of a therapeutic approach for living organisms.

The subject matter described herein provides a non-invasive, safe, convenient, and effective methodology to deliver the positive effects of far-infrared therapy to a subject. For example, in some embodiments, a patient carries, wears and/or uses the bioceramic compositions, for example when applied to an article of manufacture such as a shirt, at home and/or in the course of carrying out daily activities to help extend the benefits of the treatment the patient may receive at a clinic or to improve a patient's condition during or after physical exercise.

A feature of the subject matter described herein, including the articles, compositions of matter, methods, devices, and systems, is a composition that comprises a bioceramic, provided that when heated or exposed to heat, such as the warmth of the human body, the bioceramic provides a biomodulatory physiological effect when the article is applied to a subject. In some embodiments, the article is an apparel of clothing such as a shirt.

In some embodiments, an article comprising the bioceramic is applied to an individual undergoing treatment for cancer, such as chemotherapy or radiation therapy, such as in a shirt of blanket to be used by the subject while sleeping. In some embodiments, application of the bioceramic (or an article comprising the bioceramic) leads to an improved recovery from the treatment of cancer, such as an improved mood, sense of well-being, or a reduction in the perception of one or more side effects from the cancer treatment.

In one aspect, provided herein, is a method of improving recovery in a subject who has undergone and/or is undergoing treatment for cancer, comprising contacting an article comprising a bioceramic with the subject's body, wherein when heated or exposed to heat, the bioceramic composition provides far infrared thermal radiation to the subject, thereby providing an improvement in recovery in the subject. In some embodiments, the treatment for cancer comprises radiation therapy, chemotherapy, immunotherapy, or any combination thereof. In some embodiments, the treatment for cancer comprises chemotherapy.

In some embodiments, the article comprising the bioceramic contacts skin of the subject.

In some embodiments, the improvement comprises an improved quality of life, reduced fatigue, improved balance, improved hydration, an increase in total plasma antioxidants, improved quality of sleep, improved status of the autonomic nervous system, or any combination thereof.

In some embodiments, the improvement comprises improved quality of life as measured using the World Health Organization Quality of Life Questionnaire. In some embodiments, the improvement comprises reduced fatigue as measured by a fatigue pictogram psychometric evaluation. In some embodiments, the improvement comprises improved balance as measured by baropodometry analysis. In some embodiments, the improvement comprises improved hydration as measured by a bioimpedance analysis. In some embodiments, the improvement comprises an increase in total plasma antioxidants. In some embodiments, the improvement comprises improved quality of sleep as measured by the Pittsburgh Quality of Sleep Index. In some embodiments, the improvement comprises improved status of the autonomic nervous system. In some embodiments, the improvement comprises a reduced heart rate variability (HRV). In some embodiments, the improved status of the autonomic nervous system comprises decreased markers of sympathetic activity, increased markers of parasympathetic activity, or both.

In some embodiments, the bioceramic comprises kaolinite and tourmaline. In some embodiments, the bioceramic comprises from about 20 wt % to about 80 wt % kaolinite and from about 1 wt % to about 30 wt % tourmaline. In some embodiments, the bioceramic comprises at least one additional oxide. In some embodiments, wherein the one additional oxide comprises from about 1 to 20 wt % of the bioceramic. In some embodiments, the one additional oxide is selected from the group consisting of aluminum oxide (Al₂O₃), silicon dioxide (SiO₂), titanium dioxide (TiO₂), magnesium oxide (MgO), and zirconium dioxide (ZrO₂).

In some embodiments, the bioceramic composition comprises from about 40 wt % to about 60 wt % kaolinite (Al₂Si₂O₅(OH)₄) to about 5 wt % to about 15 wt % tourmaline to about 15 wt % to about 25 wt % aluminum oxide (Al₂O₃) to about 10 wt % to about 20 wt % silicon dioxide (SiO₂); and from about 1 wt % to about 20 wt % titanium dioxide (TiO₂); provided that the total amounts are by total weight of the composition. In some embodiments, the bioceramic composition comprises from about 40 wt % to about 60 wt % kaolinite (Al₂Si₂O₅(OH)₄) to about 5 wt % to about 15 wt % tourmaline to about 15 wt % to about 25 wt % aluminum oxide (Al₂O₃) to about 10 wt % to about 20 wt % silicon dioxide (SiO₂); and from about 1 wt % to about 20 wt % magnesium oxide (MgO); provided that the total amounts are by total weight of the composition. In some embodiments, the bioceramic composition comprises from about 40 wt % to about 60 wt % kaolinite (Al₂Si₂O₅(OH)₄) to about 5 wt % to about 15 wt % tourmaline to about 15 wt % to about 25 wt % aluminum oxide (Al₂O₃) to about 10 wt % to about 20 wt % silicon dioxide (SiO₂); and from about 1 wt % to about 20 wt % zirconium dioxide (ZrO₂); provided that the total amounts are by total weight of the composition.

In some embodiments, the article is clothing. In some embodiments, the article is shirt, a jacket, shorts or trousers.

In some embodiments, the article is bedding. In some embodiments, the bedding is selected from blankets, sheets, pillows, pillowcases, comforters, duvet covers, mattress covers, and mattress pads. In some embodiments, the bedding is a blanket.

In some embodiments, the article comprising the bioceramic is applied to the body of the subject for at least 1 hour, at least 2 hours, at least 3 hours, at least 4 hours, at least 5 hours, at least 6 hours, at least 7 hours, or at least 8 hours. In some embodiments, the article comprising the bioceramic is applied to the body of the subject daily, wherein the article comprising the bioceramic is applied to the body of the subject for at least 1 hour, at least 2 hours, at least 3 hours, at least 4 hours, at least 5 hours, at least 6 hours, at least 7 hours, or at least 8 hours each day. In some embodiments, the article comprising the bioceramic is applied to the body of the subject daily over a period of time of at least 1 week, at least 2 weeks, at least 3 weeks, at least 4 weeks, at least 5 weeks, or at least 6 weeks. In some embodiments, the article comprising the bioceramic is applied to the body of the subject daily, wherein the article comprising the bioceramic is applied to the body of the subject for at most 1 hour, at most 2 hours, at most 3 hours, at most 4 hours, at most 5 hours, at most 6 hours, at most 7 hours, or at most 8 hours each day. In some embodiments, the article comprising the bioceramic is applied to the body of the subject daily over a period of time of at most 1 week, at most 2 weeks, at most 3 weeks, at most 4 weeks, at most 5 weeks, or at most 6 weeks.

### BRIEF DESCRIPTION OF THE DRAWINGS

The novel and inventive features of the invention are set forth with particularity in the appended claims. A better understanding of the features and advantages of the present invention will be obtained by reference to the following detailed description that sets forth illustrative embodiments, in which the principles of the invention are utilized, and the accompanying drawings which in this provisional patent application are provided in the Examples section below.

FIG. 1 illustrates a non-limiting example of a shirt comprising a bioceramic of the instant disclosure.

FIG. 2 illustrates a non-limiting example of a shirt and a pad comprising a bioceramic of the instant disclosure.

FIG. 3 illustrates a non-limiting example of the infrared transmittance of distinct bioceramic compositions of the instant disclosure. FIG. 3A illustrates the infrared transmittance of a bioceramic compositions described herein comprising 18% aluminium oxide, 14% silicon dioxide, 50% kaolinite, 8% zirconium oxide, and 10% tourmaline. FIG. 3B illustrates the infrared transmittance of a bioceramic compositions described herein comprising 20% aluminum, 3% titanium, 11% magnesium oxide, 6% diiron trioxide, and 60% silica.

FIG. 4 shows results from Quality of Life surveys taken by cancer patients before treatment with bioceramic and after six weeks of treatment with a bioceramic.

FIG. 5 shows results from fatigue pictogram psychometric evaluations of cancer patients before treatment with bioceramic and after six weeks of treatment with bioceramic.

FIG. 6 shows results from balance baropodometry platform evaluations of cancer patients before treatment with bioceramic and after six weeks of treatment with bioceramic.

FIG. 7 shows results from hydration bioimpedance analysis of cancer patients before treatment with bioceramic and after six weeks of treatment with bioceramic.

FIG. 8 shows results from hydration bioimpedance analysis of cancer patients before treatment with bioceramic and after six weeks of treatment with bioceramic.

FIG. 9 shows results from plasma antioxidant measurements of cancer patients before treatment with bioceramic and after six weeks of treatment with bioceramic.

FIG. 10 shows results from Pittsburgh Quality of Sleep Index evaluations of cancer patients before treatment with bioceramic and after six weeks of treatment with bioceramic.

FIG. 11 shows results from Heart Rate Variability (HRV) analysis of cancer patients before treatment with bioceramic and after six weeks of treatment with bioceramic.

FIG. 12 shows results from Heart Rate Variability (HRV) analysis of cancer patients before treatment with bioceramic and after six weeks of treatment with bioceramic.

### DETAILED DESCRIPTION OF THE INVENTION

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.”

Without being limited by theory the instant inventors have discovered that the biological effects of bioceramics are based on the fact that the infrared frequency range is the natural resonant frequency range of water and living organisms. Because a considerable part of living organisms include water, the resonant frequency of water molecules radiated from the bioceramics described herein can activate the water and affect living organisms, including humans, and including the treatment of disease and biological complications and pathways.

The bioceramics of the disclosure radiate far infrared energy towards the body or away from the body of a subject. When a bioceramic radiates energy towards the body of a subject, the bioceramic provides concentrated radiant energy to cells by reflecting the far infrared energy or rays of the body heat into the subject's joints, muscles, and tissues. The far infrared energy penetrates the cells and provides biomodulatory or physiological effects, such as anti-inflammatory, analgesic, and other biomodulatory or physiological effects. When a bioceramic radiates energy away from the body of a subject, the bioceramic prevents far infrared energy from penetrating the skin of a subject, thereby providing a cooling effect.

### Bioceramic Compositions

An aspect of the articles, compositions of matter, methods, devices, and systems described herein is a bioceramic composition that in certain applications provides improved recovery to a patient undergoing a cancer therapy. For example, in some embodiments, provided is a bioceramic composition that when heated or exposed to heat provides the improved or physiological effect when the article is applied to a subject. In some instances, the disclosed bioceramic formulations are pre-mixed and prepared as a powder formulation. In other instances, the individual components of a bioceramic can be separately added to a substrate. In some instances, the bioceramic formulations described herein generally comprise from about 20 wt % to about 80 wt % kaolinite (Al₂Si₂O₅(OH)₄), from about 1 wt % to about 30 wt % tourmaline, and at least one additional oxide from about 1 to 20% wt %, provided that the amounts are by total weight of the bioceramic composition. The at least one additional oxide can be selected from the group consisting of aluminum oxide (Al₂O₃), silicon dioxide (SiO₂), titanium dioxide (TiO₂), magnesium oxide (MgO), and zirconium dioxide (ZrO₂).

In some instances, the bioceramic composition comprises from about 40 wt % to about 60 wt % kaolinite (Al₂Si₂O₅(OH)₄) to about 5 wt % to about 15 wt % tourmaline to about 15 wt % to about 25 wt % aluminum oxide (Al₂O₃) to about 10 wt % to about 20 wt % silicon dioxide (SiO₂); and from about 1 wt % to about 20 wt % titanium dioxide (TiO₂); provided that the total amounts are by total weight of the composition.

In other instances, the bioceramic composition comprises from about 40 wt % to about 60 wt % kaolinite (Al₂Si₂O₅(OH)₄) to about 5 wt % to about 15 wt % tourmaline to about 15 wt % to about 25 wt % aluminum oxide (Al₂O₃) to about 10 wt % to about 20 wt % silicon dioxide (SiO₂); and from about 1 wt % to about 20 wt % magnesium oxide (MgO); provided that the total amounts are by total weight of the composition.

In some instances, the bioceramic composition comprises from about 40 wt % to about 60 wt % kaolinite (Al₂Si₂O₅(OH)₄) to about 5 wt % to about 15 wt % tourmaline to about 15 wt % to about 25 wt % aluminum oxide (Al₂O₃) to about 10 wt % to about 20 wt % silicon dioxide (SiO₂); and from about 1 wt % to about 20 wt % zirconium dioxide (ZrO₂); provided that the total amounts are by total weight of the composition.

In one embodiment, the bioceramic comprises:
a. about 20 wt % to about 80 wt % kaolinite (Al₂Si₂O₅(OH)₄);
b. about 1 wt % to about 30 wt % tourmaline;
c. about 1 wt % to about 40 wt % aluminum oxide (Al₂O₃);
d. about 1 wt % to about 40 wt % silicon dioxide (SiO₂); and
e. about 1 wt % to about 20 wt % one additional oxide; provided that the amounts are by total weight of the bioceramic composition. In some embodiments, the one additional oxide is selected from the group consisting of titanium dioxide (TiO₂), magnesium oxide (MgO), and zirconium dioxide (ZrO₂). In some embodiments, the one additional oxide is TiO₂.

In further or additional embodiments, provided is a bioceramic composition of matter that when heated or exposed to heat provides a biomodulatory or physiological effect when the article is applied to a subject, comprising:
a. about 40 wt % to about 60 wt % kaolinite (Al₂Si₂O₅(OH)₄);
b. about 5 wt % to about 15 wt % tourmaline;
c. about 15 wt % to about 25 wt % aluminum oxide (Al₂O₃);
d. about 10 wt % to about 20 wt % silicon dioxide (SiO₂); and
e. about 1 wt % to about 20 wt % one additional oxide; provided that the amounts are by total weight of the bioceramic composition. In some embodiments, the additional oxide is a metal oxide. In some embodiments, the one additional oxide is selected from the group consisting of titanium dioxide (TiO₂), magnesium oxide (MgO), and zirconium dioxide (ZrO₂). In some embodiments, the one additional oxide is TiO₂. In some embodiments, the bioceramic composition comprises kaolinite in a range from about 45 wt % to about 55 wt %. In further or additional embodiments, provided is a bioceramic composition that comprises kaolinite in the range from about 47 wt % to about 53 wt %. In further or additional embodiments, provided is a bioceramic composition that contains kaolinite in a range from about 48 wt % to about 52 wt %. In some embodiments, the bioceramic comprises about 7 wt % to about 13 wt % tourmaline. In some embodiments, the bioceramic comprises about 8 wt % to about 12 wt % tourmaline. In some embodiments, the bioceramic comprises about 17 wt % to about 23 wt % aluminum oxide. In some embodiments, the bioceramic comprises about 18 wt % to about 22 wt % aluminum oxide. In some embodiments, the bioceramic comprises about 12 wt % to about 18 wt % silicon dioxide. In some embodiments, the bioceramic comprises about 13 wt % to about 17 wt % silicon dioxide. In some embodiments, the bioceramic comprises about 5 wt % to about 15 wt % of the additional oxide. In some embodiments, the bioceramic comprises about 5 wt % to about 12 wt % of the additional oxide. In some embodiments, the bioceramic comprises about 5 wt % to about 10 wt % of the additional oxide. In some embodiments, the additional oxide is a metal oxide. In some embodiments, the one additional oxide is selected from the group consisting of titanium dioxide (TiO₂), magnesium oxide (MgO), and zirconium dioxide (ZrO₂). In some embodiments, the one additional oxide is TiO₂.

In some embodiments, provided is a bioceramic composition that comprises
a. about 50 wt % kaolinite (Al₂Si₂O₅(OH)₄);
b. about 10 wt % tourmaline;
c. about 18 wt % aluminum oxide (Al₂O₃);
d. about 14 wt % silicon dioxide (SiO₂); and
e. about 8 wt % one additional oxide; provided that the amounts are by total weight of the bioceramic composition. In some embodiments, the one additional oxide is selected from the group consisting of titanium dioxide (TiO₂), magnesium oxide (MgO), and zirconium dioxide (ZrO₂). In some embodiments, the one additional oxide is TiO₂.

Another feature of the subject matter described herein are bioceramic compositions that include tourmaline. As used herein, the term “tourmaline” retains its meaning known in the mineral and gemstone arts. For example, tourmaline, is a group of isomorphous minerals with an identical crystal lattice. Each member of the tourmaline group has its own chemical formula, due to small differences in their elemental distribution. For example, in some embodiments, the tourmaline has the following generic formula X₁Y₃Al₆(BO₃)₃Si₆O₁₈(OH)₄, where: X=Na and/or Ca and Y=Mg, Li, Al, and/or Fe²⁺, which is represented with the following formula, (Na,Ca)(Mg,Li,A,Fe²⁺)₃Al₆(BO₃)₃Si₆O₁₈(OH)₄.

In some embodiments, the Al may be replaced by other elements. For example, in Uvite, the Al is partially replaced by Mg which expands the formula to: (Na,Ca)(Mg,Li,Al,Fe²⁺)₃ (Al,Mg,Cr)₆(BO₃)₃Si₆O₁₈(OH)₄.

In some embodiments, the tourmaline is Buergerite which contains three O atoms and one F atom in place of the OH radical. A Buergerite molecule also contains an Fe atom that is in a 3+ oxidation state which is depicted as: (Na,Ca)(Mg,Li,A,Fe²⁺,Fe³⁺)₃(Al,Mg,Cr)₆(BO₃)₃Si₆O₁₈(OH,O,F)₄. In other embodiments, the tourmaline is one or more of the following:
Schorl: NaFe²⁺₃Al₆(BO₃)₃Si₆O₁₈(OH)₄;
Dravite: NaMg₃Al₆(BO₃)₃Si₆O₁₈(OH)₄;
Elbaite: Na(Li,Al)₃Al₆(BO₃)₃Si₆O₁₈(OH)₄;
Liddicoatite: Ca(Li,Al)₃Al₆(BO₃)₃Si₆O₁₈(OH)₄;
Uvite: Ca(Mg,Fe²⁺)₃Al₅Mg(BO₃)₃Si₆O₁₈(OH)₄;
Buergerite: NaFe³⁺₃Al₆(BO₃)₃Si₆O₁₈O₃F.
   In one embodiment, the bioceramic composition tourmaline that comprises NaFe²⁺₃Al₆Si₆O₁₈(BO₃)₃(OH)₃OH.

Another aspect of the articles, compositions of matter, methods, devices, and systems described herein is a bioceramic composition of micrometer particle size. For example, in some embodiments, provided is a bioceramic composition containing a largest dimension of any particle in the bioceramic of from about 0.1 micrometer (μm) to about 250 micrometers. In further or additional embodiments, provided is a bioceramic composition, provided that the largest dimension of any particle in the bioceramic is from about 0.5 micrometers to about 25 micrometers. In some cases, a bioceramic particle can have a diameter, or cross-sectional area, of about 0.1 μm to about 1 μm, of about 0.1 μm to about 10 μm, of about 0.1 μm to about 20 μm, of about 0.1 μm to about 30 μm, of about 0.1 μm to about 40 μm, of about 0.1 μm to about 50 μm, of about 0.1 μm to about 60 μm, of about 0.1 μm to about 70 μm, of about 0.1 μm to about 80 μm, of about 0.1 μm to about 90 μm, of about 0.1 μm to about 100 μm, or other desired size. In some cases, an inlet can have a cross-sectional diameter of about 10 μm to about 100 μm, of about 10 μm to about 200 m, of about 10 m to about 300 m, of about 10 m to about 400 m, of about 10 μm to about 500 μm, or other desired size.

In further or additional embodiments, provided is a bioceramic composition of matter that when heated or exposed to heat provides a biomodulatory or physiological effect when the article is applied to a subject, wherein the bioceramic composition comprises tourmaline, kaolinite and at least one oxide. In some cases a bioceramic of the disclosure comprises tourmaline, kaolinite, aluminum oxide and silicon dioxide. In some cases a bioceramic of the disclosure comprises tourmaline, kaolinite, aluminum oxide, silicon dioxide and one other oxide. In some cases, the other oxide is zirconium oxide. In some cases the other oxide is titanium dioxide (TiO₂). In some cases the other oxide is magnesium oxide (MgO).

Kaolinite is a layered silicate mineral comprising oxides. In some cases, various oxides are comprised within the kaolinite. In some cases, a bioceramic composition comprises additional oxides that are not part of the kaolinite. In some embodiments, a bioceramic composition comprises one oxide, two oxides, three oxides, four oxides, five oxides, six oxides, seven oxides, eight oxides, nine oxides, ten oxides, eleven oxides, twelve oxides, or more oxides. In some cases, the additional oxides are highly refractory oxides. [0047].

In some embodiments, an oxide of a bioceramic composition of matter of the disclosure has various oxidation states. An oxide of the disclosure has an oxidation number of +1, +2, +3, +4, +5, +6, +7, or +8. In some cases a bioceramic composition of the disclosure will have more than one oxide wherein at least one oxide has a different oxidation number as compared to the other oxide. For example, in some cases a bioceramic composition of the disclosure comprises an aluminum oxide (Al₂O₃) with a +2 or a +3 oxidation state, a silicon dioxide (SiO₂) with a +4 oxidation state, and a zirconium oxide (ZrO₂) with a +4 oxidation state.

In some cases a bioceramic of the disclosure comprises tourmaline, kaolinite, aluminum oxide and silicon dioxide. In some cases a bioceramic of the disclosure comprises tourmaline, kaolinite, aluminum oxide, silicon dioxide and one other oxide. In some embodiments, the other oxide is an oxide with a +1, +2, +3, +4, +5, +6, or +7 oxidation state.

Non-limiting examples of oxides with +1 oxidation state include: copper(I) oxide (Cu₂O), dicarbon monoxide (C₂O), dichlorine monoxide (Cl₂O), lithium oxide (Li₂O), potassium oxide (K₂O), rubidium oxide (Rb₂O), silver oxide (Ag₂O), thallium(I) oxide (Tl₂O), sodium oxide (Na₂O), or water (Hydrogen oxide) (H₂O).

Non-limiting examples of oxides with +2 oxidation state include: aluminium(II) oxide (AlO), barium oxide (BaO), beryllium oxide (BeO), cadmium oxide (CdO), calcium oxide (CaO), carbon monoxide (CO), chromium(II) oxide (CrO), cobalt(II) oxide (CoO), copper(II) oxide (CuO), iron(II) oxide (FeO), lead(II) oxide (PbO), magnesium oxide (MgO), mercury(II) oxide (HgO), nickel(II) oxide (NiO), nitric oxide (NO), palladium(II) oxide (PdO), strontium oxide (SrO), sulfur monoxide (SO), disulfur dioxide (S₂O₂), tin(II) oxide (SnO), titanium(II) oxide (TiO), vanadium(II) oxide (VO), or zinc oxide (ZnO).

Non-limiting examples of oxides with +3 oxidation states include: aluminium oxide (Al₂O₃), antimony trioxide (Sb₂O₃), arsenic trioxide (As₂O₃), bismuth(III) oxide (Bi₂O₃), boron trioxide (B₂O₃), chromium(III) oxide (Cr₂O₃), dinitrogen trioxide (N₂O₃), erbium(III) oxide (Er₂O₃), gadolinium(III) oxide (Gd₂O₃), gallium(III) oxide (Ga₂O₃), holmium(III) oxide (Ho₂O₃) indium(III) oxide (In₂O₃), iron(III) oxide (Fe₂O₃), lanthanum oxide (La₂O₃), lutetium(III) oxide (Lu₂O₃), nickel(III) oxide (Ni₂O₃), phosphorus trioxide (P₄O₆), promethium(III) oxide (Pm₂O₃), rhodium(III) oxide (Rh₂O₃), samarium(III) oxide (Sm₂O₃), scandium oxide (Sc₂O₃), terbium(III) oxide (Tb₂O₃), thallium(III) oxide (Tl₂O₃), thulium(III) oxide (Tm₂O₃), titanium(III) oxide (Ti₂O₃), tungsten(III) oxide (W₂O₃), vanadium(III) oxide (V₂O₃), ytterbium(III) oxide (Yb₂O₃), yttrium(III) oxide (Y₂O₃).

Non-limiting examples of oxides with +4 oxidation states include: carbon dioxide (CO₂), carbon trioxide (CO₃), cerium(IV) oxide (CeO₂), chlorine dioxide (ClO₂), chromium(IV) oxide (CrO₂), dinitrogen tetroxide (N₂O₄), germanium dioxide (GeO₂), hafnium(IV) oxide (HfO₂), lead dioxide (PbO₂), manganese dioxide (MnO₂), nitrogen dioxide (NO2), plutonium(IV) oxide (PuO₂), rhodium(IV) oxide (RhO₂), ruthenium(IV) oxide (RuO₂), selenium dioxide (SeO₂), silicon dioxide (SiO₂), sulfur dioxide (SO₂), tellurium dioxide (TeO₂), thorium dioxide (ThO₂), tin dioxide (SnO₂), titanium dioxide (TiO₂), tungsten(IV) oxide (WO₂), uranium dioxide (UO₂), vanadium(IV) oxide (VO₂), or zirconium dioxide (ZrO₂).

Non-limiting examples of oxides with +5 oxidation states include: antimony pentoxide (Sb₂O₅), arsenic pentoxide (As₂O₅), dinitrogen pentoxide (N₂O₅), niobium pentoxide (Nb₂O₅), phosphorus pentoxide (P₂O₅), tantalum pentoxide (Ta₂O₅), or vanadium(V) oxide (V₂O₅). Non-limiting examples of oxides with +6 oxidation states include: chromium trioxide (Cr₀₃), molybdenum trioxide (MoO₃), rhenium trioxide (ReO₃), selenium trioxide (SeO₃), sulfur trioxide (SO₃), tellurium trioxide (TeO₃), tungsten trioxide (WO₃), uranium trioxide (UO₃), or xenon trioxide (XeO₃).

Non-limiting examples of oxides with +7 oxidation states include: dichlorine heptoxide (Cl₂O₇), manganese heptoxide (Mn₂O₇), rhenium(VII) oxide (Re₂O₇), or technetium(VII) oxide (Tc₂O₇). Non-limiting examples of oxides with +8 oxidation states include: osmium tetroxide (OsO₄), ruthenium tetroxide (RuO₄), xenon tetroxide (XeO₄), iridium tetroxide (IrO₄), or hassium tetroxide (HsO₄). Non-limiting examples of oxides with various states of oxidation include antimony tetroxide (Sb₂O₄), cobalt(II,III) oxide (Co₃O₄), iron(II,III) oxide (Fe₃O₄), lead(II,IV) oxide (Pb₃O₄), manganese(II,III) oxide (Mn₃O₄), or silver(I,III) oxide (AgO).

In further or additional embodiments a bioceramic composition of matter of the disclosure further comprises a metal. A metal can be in elemental form, such as a metal atom, or a metal ion. Non-limiting examples of metals include transition metals, main group metals, and metals of Group 3, Group 4, Group 5, Group 6, Group 7, Group 8, Group 9, Group 10, Group 11, Group 12, Group 13, Group 14, and Group 15 of the Periodic Table. Non-limiting examples of metal include scandium, titanium, vanadium, chromium, manganese, iron, cobalt, nickel, copper, zinc, yttrium, zirconium, niobium, molybdenum, technetium, ruthenium, rhodium, palladium, silver, cadmium, lanthanum, hafnium, tantalum, tungsten, rhenium, osmium, iridium, platinum, gold, mercury, tin, lead, and bismuth.

The proportion of minerals and oxides in a bioceramic composition can optionally be altered depending on a number of variables, including, for example, the amount of thermal radiation, more specifically far infrared radiation, to be emitted, the disease or condition to be treated, the mode of administration, the requirements of the individual subject, the severity of the disease or condition being treated, or the judgment of a practitioner.

### Physical Properties

Tourmaline and kaolinite have distinct granulometric, mineralogical, chemical, and physical properties depending on, for example, whether the minerals are extracted from a particular geographic region or whether the minerals are chemically synthesized. For instance, in many parts of the world a kaolinite has a pink-orange-red coloration that is associated with an amount of an impurity(ies). Often, the impurity(ies) comprises iron oxide. In some embodiments, a kaolinite of the disclosure is of a high purity level, and it is characterized by a fine white color.

In some embodiments, a purity of the tourmaline or kaolinite is associated with an amount of infrared energy that is radiated from a bioceramic composition. In some cases the kaolinite or tourmaline of a bioceramic composition of the disclosure is greater than 99% pure, greater than 98% pure, greater than 97% pure, greater than 96% pure, greater than 95% pure, greater than 94% pure, greater than 93% pure, greater than 92% pure, greater than 91% pure, greater than 90% pure, greater than 89% pure, greater than 88% pure, greater than 87% pure, greater than 86% pure, greater than 85% pure, greater than 80% pure, greater than 75% pure, greater than 70% pure, greater than 65% pure, greater than 60% pure, or greater than 55% pure.

In some embodiments, a granularity of a kaolinite or tourmaline is associated with an amount of infrared energy that is radiated from a bioceramic composition. For instance, a bioceramic composition comprising coarser-size mineral reflects a different amount of infrared energy as compared to a bioceramic composition comprising finer-size minerals. In some embodiments, the granularity of a bioceramic composition ranges from about 100 nanometers to about 0.1 micrometers, from about 100 nanometers to about 1 micrometer, from about 100 nanometers to about 10 micrometers, from about 100 nanometers to about 25 micrometers, from about 100 nanometers to about 50 micrometers, from about 100 nanometers to about 75 micrometers, from about 100 nanometers to about 100 micrometers, from about 100 nanometers to about 125 micrometers, from about 100 nanometers to about 150 micrometers, from about 100 nanometers to about 175 micrometers, from about 100 nanometers to about 200 micrometers, from about 100 nanometers to about 225 micrometers, or from about 100 nanometers to about 250 micrometers.

In some embodiments, the granularity of a bioceramic composition ranges from about 0.5 micrometers to about 1 micrometer, from about 0.5 micrometers to about 10 micrometers, from about 0.5 micrometers to about 25 micrometers, from about 0.5 micrometers to about 50 micrometers, from about 0.5 micrometers to about 75 micrometers, from about 0.5 micrometers to about 100 micrometers, from about 0.5 micrometers to about 125 micrometers, from about 0.5 micrometers to about 150 micrometers, from about 0.5 micrometers to about 175 micrometers, from about 0.5 micrometers to about 200 micrometers, from about 0.5 micrometers to about 225 micrometers, or from about 0.5 micrometers to about 250 micrometers.

### Far-Infrared Emittance, Transmission, and Reflection

Yet another aspect of the articles, compositions of matter, methods, devices, and systems described herein is a bioceramic composition that emits, transmits, and/or reflects an infrared wavelength when heated or exposed to heat. In some embodiments, provided is a bioceramic. In some embodiments, provided is a bioceramic that absorbs, stores, and/or reflects thermal energy, such as far infrared energy or rays. In some embodiments, provided is a bioceramic that emits, transmits, or reflects an infrared wavelength that is far infrared and that comprises a wavelength from about 1 micrometer to about 1 millimeter. In further or additional embodiments, provided is a bioceramic composition that emits, transmits, or reflects an infrared wavelength that is from about 3 micrometers to about 15 micrometers. In further or additional embodiments, described herein is a bioceramic composition that provides a reflectance of the bioceramic at a room temperature of 25° C. is at least 80% in an infrared range between about 7 micrometers and about 12 micrometers.

The material emissivity of a bioceramic material can be measured with, for example, a calorimeter or a Flir thermographic camera. A calorimeter can be used to measure the amount of thermal energy that can be received, store, and/or release by an apparel comprising a bioceramic. A Flir thermographic camera can create a thermal image of various types of apparel comprising a bioceramic of the disclosure. A Flir thermographic camera can detect up to thousands of measurement points in each thermal image and provide emissivity data for each image.

A bioceramic composition of the disclosure is formulated to have desired refractory properties. In some embodiments a bioceramic of the disclosure reflects about 99% of the infrared energy or rays received, about 98% of the infrared energy or rays received, about 97% of the infrared energy or rays received, about 96% of the infrared energy or rays received, about 95% of the infrared energy or rays received, about 94% of the infrared energy or rays received, about 93% of the infrared energy or rays received, about 92% of the infrared energy or rays received, about 91% of the infrared energy or rays received, about 90% of the infrared energy or rays received, about 89% of the infrared energy or rays received, about 88% of the infrared energy or rays received, about 87% of the infrared energy or rays received, about 86% of the infrared energy or rays received, about 85% of the infrared energy or rays received, about 84% of the infrared energy or rays received, about 83% of the infrared energy or rays received, about 82% of the infrared energy or rays received, about 81% of the infrared energy or rays received, about 80% of the infrared energy or rays received, about 79% of the infrared energy or rays received, about 78% of the infrared energy or rays received, about 77% of the infrared energy or rays received, about 76% of the infrared energy or rays received, about 75% of the infrared energy or rays received, about 74% of the infrared energy or rays received, about 73% of the infrared energy or rays received, about 72% of the infrared energy or rays received, about 71% of the infrared energy or rays received, about 70% of the infrared energy or rays received, about 65% of the infrared energy or rays received, about 60% of the infrared energy or rays received, about 55% of the infrared energy or rays received, about 50% of the infrared energy or rays received, about 45% of the infrared energy or rays received, about 40% of the infrared energy or rays received, about 35% of the infrared energy or rays received, about 30% of the infrared energy or rays received, about 25% of the infrared energy or rays received, about 20% of the infrared energy or rays received, about 15% of the infrared energy or rays received, about 10% of the infrared energy or rays received, or about 5% of the infrared energy or rays received.

In some cases a bioceramic of the disclosure reflects greater than 99% of the infrared energy or rays received, greater than 98% of the infrared energy or rays received, greater than 97% of the infrared energy or rays received, greater than 96% of the infrared energy or rays received, greater than 95% of the infrared energy or rays received, greater than 94% of the infrared energy or rays received, greater than 93% of the infrared energy or rays received, greater than 92% of the infrared energy or rays received, greater than 91% of the infrared energy or rays received, greater than 90% of the infrared energy or rays received, greater than 89% of the infrared energy or rays received, greater than 88% of the infrared energy or rays received, greater than 87% of the infrared energy or rays received, greater than 86% of the infrared energy or rays received, greater than 85% of the infrared energy or rays received, greater than 84% of the infrared energy or rays received, greater than 83% of the infrared energy or rays received, greater than 82% of the infrared energy or rays received, greater than 81% of the infrared energy or rays received, greater than 80% of the infrared energy or rays received, greater than 79% of the infrared energy or rays received, greater than 78% of the infrared energy or rays received, greater than 77% of the infrared energy or rays received, greater than 76% of the infrared energy or rays received, greater than 75% of the infrared energy or rays received, greater than 74% of the infrared energy or rays received, greater than 73% of the infrared energy or rays received, greater than 72% of the infrared energy or rays received, greater than 71% of the infrared energy or rays received, greater than 70% of the infrared energy or rays received, greater than 65% of the infrared energy or rays received, greater than 60% of the infrared energy or rays received, greater than 55% of the infrared energy or rays received, greater than 50% of the infrared energy or rays received, greater than 45% of the infrared energy or rays received, greater than 40% of the infrared energy or rays received, greater than 35% of the infrared energy or rays received, greater than 30% of the infrared energy or rays received, greater than 25% of the infrared energy or rays received, greater than 20% of the infrared energy or rays received, greater than 15% of the infrared energy or rays received, greater than 10% of the infrared energy or rays received, or greater than 5% of the infrared energy or rays received.

In some cases a bioceramic of the disclosure reflects fewer than 99% of the infrared energy or rays received, fewer than 98% of the infrared energy or rays received, fewer than 97% of the infrared energy or rays received, fewer than 96% of the infrared energy or rays received, fewer than 95% of the infrared energy or rays received, fewer than 94% of the infrared energy or rays received, fewer than 93% of the infrared energy or rays received, fewer than 92% of the infrared energy or rays received, fewer than 91% of the infrared energy or rays received, fewer than 90% of the infrared energy or rays received, fewer than 89% of the infrared energy or rays received, fewer than 88% of the infrared energy or rays received, fewer than 87% of the infrared energy or rays received, fewer than 86% of the infrared energy or rays received, fewer than 85% of the infrared energy or rays received, fewer than 84% of the infrared energy or rays received, fewer than 83% of the infrared energy or rays received, fewer than 82% of the infrared energy or rays received, fewer than 81% of the infrared energy or rays received, fewer than 80% of the infrared energy or rays received, fewer than 79% of the infrared energy or rays received, fewer than 78% of the infrared energy or rays received, fewer than 77% of the infrared energy or rays received, fewer than 76% of the infrared energy or rays received, fewer than 75% of the infrared energy or rays received, fewer than 74% of the infrared energy or rays received, fewer than 73% of the infrared energy or rays received, fewer than 72% of the infrared energy or rays received, fewer than 71% of the infrared energy or rays received, fewer than 70% of the infrared energy or rays received, fewer than 65% of the infrared energy or rays received, fewer than 60% of the infrared energy or rays received, fewer than 55% of the infrared energy or rays received, fewer than 50% of the infrared energy or rays received, fewer than 45% of the infrared energy or rays received, fewer than 40% of the infrared energy or rays received, fewer than 35% of the infrared energy or rays received, fewer than 30% of the infrared energy or rays received, fewer than 25% of the infrared energy or rays received, fewer than 20% of the infrared energy or rays received, fewer than 15% of the infrared energy or rays received, fewer than 10% of the infrared energy or rays received, or fewer than 5% of the infrared energy or rays received.

In some embodiments, the bioceramic reflects far infrared energy towards the body of a subject and in some embodiments the bioceramic reflects far infrared energy away from the body of the subject. A bioceramic can provide a cooling effect when it reflects infrared energy away from the body. In some embodiments a bioceramic is adjacent to or near an insulator. In some embodiments, an article comprising an insulated bioceramic provides a cooling effect to a subject, provided that when heated or exposed to heat, the bioceramic reflects the far infrared rays away from the subject.

In some embodiments, an apparel of the disclosure comprises an insulator that is in contact with or is adjacent to a bioceramic. The insulator can be used in embodiments where the apparel comprising the bioceramic is fabricated to reflect far infrared energy away from the body of a subject. In some embodiments, the insulator is a material of low thermal conductivity and prevents far infrared energy from being reflected in a direction. Different types of materials can be used to reflect infrared, non-limiting examples of insulators include rubber, glass, paper, plastic, wood, cloth, foil, or styrofoam.

An apparel of the disclosure can provide a therapeutically-effective amount of infrared to a subject. In some cases the apparel is a shirt comprising a bioceramic, and when exposed to heat, the shirt comprising the bioceramic provides at least 1.5 joules/cm² of far infrared rays to a subject. In some cases the apparel is athletic apparel, a sporting accessory, or a sports equipment including, but not limited to, orthotic inserts, athletic shoes, diving suits, life preservers, shirts, shorts, wrist bands, arm bands, head bands, gloves, jackets, pants, hats, and backpacks, skis, ski poles, snowboards, skateboards, in-line skates, bicycles, surf boards, water skis, jet skis, diving equipment, ropes, chains, goggles, and/or blankets. In some embodiments, the apparel is a sporting accessory, including but not limited to a blanket. In some embodiments, the apparel is configured for use in orthotic applications, including but not limited to orthotic inserts, shoes, and the like. In some cases the apparel is a patch (e.g. a patch that is fabricated to adhere to skin or not, such as transdermal patches, transdermal hydrogel patches, etc.), adhesive tape, such as kinesio, non-adhesive tape, pads, insoles, bedding, including a sheet, a mattress, a cover, a pillow, and/or a pillow case, a body support, a foam roller, a lotion, a soap, tape, glassware, furniture, paint, ink, a label, carpet, a mat, a food and/or beverage container, a drink koozie (e.g. bottle or can), headware (e.g. a helmet, a hat, etc.), footwear (e.g. a shoe, sneaker, sandal, etc.), an earphone, a surface, a sports surface, an artificial grass, and the like. In some cases, the apparel is a shirt, a pant, a short, dresses, a skirt, jacket, a hat, an undergarment, a sock, a cap, a glove, a scarf, a diaper, a blanket, a comforter, a duvet cover, a mattress cover, a mattress pad, and the like. In some embodiments, the apparel is a blanket. In another embodiment, the article is a body support selected from a knee wrap, an elbow support, a compression arm sleeve, a compression leg sleeve, a wrist wrap, and the like.

In some embodiments, the subject matter described herein provides from 1 joule/cm² to 45 joules/cm², from 2-10 joules/cm², or from 4-6 joules/cm² of far infra-red energy rays or rays to a subject. In certain embodiments, the bioceramic formulation that provides at least 1 joule/cm², 1.5 joules/cm², at least 2 joules/cm², at least 3 joules/cm², at least 4 joules/cm², at least 5 joules/cm², at least 6 joules/cm², at least 7 joules/cm², at least 8 joules/cm², at least 9 joules/cm², at least 10 joules/cm², at least 11 joules/cm², at least 12 joules/cm², at least 13 joules/cm², at least 14 joules/cm², at least 15 joules/cm², at least 16 joules/cm², at least 17 joules/cm², at least 18 joules/cm², at least 19 joules/cm², at least 20 joules/cm², at least 21 joules/cm², at least 22 joules/cm², at least 23 joules/cm², at least 24 joules/cm², at least 25 joules/cm², at least 26 joules/cm², at least 27 joules/cm², at least 28 joules/cm², at least 29 joules/cm², at least 30 joules/cm², at least 31 joules/cm², at least 32 joules/cm², at least 33 joules/cm², at least 34 joules/cm², at least 35 joules/cm², at least 36 joules/cm², at least 37 joules/cm², at least 38 joules/cm², at least 39 joules/cm², at least 40 joules/cm², at least 41 joules/cm², at least 42 joules/cm², at least 43 joules/cm², at least 44 joules/cm², or about 45 joules/cm² of far infrared energy or rays to a subject.

In some cases, an apparel of the disclosure can provide at most 1.5 joules/cm², at most 2 joules/cm², at most 3 joules/cm², at most 4 joules/cm², at most 5 joules/cm², at most 6 joules/cm², at most 7 joules/cm², at most 8 joules/cm², at most 9 joules/cm², at most 10 joules/cm², at most 11 joules/cm², at most 12 joules/cm², at most 13 joules/cm², at most 14 joules/cm², at most 15 joules/cm², at most 16 joules/cm², at most 17 joules/cm², at most 18 joules/cm², at most 19 joules/cm², at most 20 joules/cm², at most 21 joules/cm², at most 22 joules/cm², at most 23 joules/cm², at most 24 joules/cm², at most 25 joules/cm², at most 26 joules/cm², at most 27 joules/cm², at most 28 joules/cm², at most 29 joules/cm², at most 30 joules/cm², at most 31 joules/cm², at most 32 joules/cm², at most 33 joules/cm², at most 34 joules/cm², at most 35 joules/cm², at most 36 joules/cm², at most 37 joules/cm², at most 38 joules/cm², at most 39 joules/cm², at most 40 joules/cm², at most 41 joules/cm², at most 42 joules/cm², at most 43 joules/cm², at most 44 joules/cm², or at most 45 joules/cm² of far infrared energy or rays to a subject.

In some cases, an apparel of the disclosure provides between 1.5 joules/cm² and 45 joules/cm², between 1.5 joules/cm² and 40 joules/cm², between 1.5 joules/cm² and 35 joules/cm², between 1.5 joules/cm² and 30 joules/cm², between 1.5 joules/cm² and 25 joules/cm², between 1.5 joules/cm² and 20 joules/cm², between 1.5 joules/cm² and 15 joules/cm², between 1.5 joules/cm² and 10 joules/cm², between 1.5 joules/cm² and 5 joules/cm², between 2 joules/cm² and 45 joules/cm², between 2 joules/cm² and 40 joules/cm², between 2 joules/cm² and 35 joules/cm², between 2 joules/cm² and 30 joules/cm², between 2 joules/cm² and 25 joules/cm², between 2 joules/cm² and 20 joules/cm², between 2 joules/cm² and 15 joules/cm², between 2 joules/cm² and 10 joules/cm², between 2 joules/cm² and 5 joules/cm² of far infrared energy or rays to a subject. In some cases, the apparatus is a shirt, and the shirt provides at most 45 joules/cm² of far infrared energy or rays to a subject.

Infrared energy can be absorbed, reflected, or emitted by molecules. In many cases, the thermal radiation emitted by objects on or near room temperature (approximately 25° C.) is infrared.

For example, in certain applications of the subject matter described herein, infrared energy is emitted or absorbed by molecules upon a rotational and/or vibrational movements. In certain embodiments, the bioceramic materials provided herein provides infrared energy elicits vibrational modes in a molecule through a change in the dipole moment. In some embodiments, absorption of heat by a bioceramic of the instant disclosure elicits vibrational modes in at least one molecule of the bioceramic through changes in the dipole moment. Further, infrared energy from the thermal radiation, in certain embodiments, is absorbed and reflected by molecules in the bioceramic when they change their rotational-vibrational energy. In further or additional embodiments, provided herein is a bioceramic that comprises a formulation of a ceramic material and vibrational technology that provides enhanced bio-modulatory properties when in contact with or applied to a subject, including as one example a human subject.

### Articles

An aspect of the articles, compositions of matter, methods, devices, and systems described herein is an article comprising a composition that comprises a bioceramic, provided that when heated or exposed to heat, the bioceramic provides a biomodulatory or physiological effect when the article is applied to a subject.

In some embodiments, provided are articles that incorporate a bioceramic composition, and articles with bioceramics applied to them. In one embodiment, the bioceramic composition is present as a coating on at least a portion of the surface of the article (for example on the inside or the outside of the article) or is incorporated directly into a substrate prior to or during manufacture of the article itself. In another embodiment, the substrate is a polymeric, cloth, or metallic material.

In some embodiments, provided are bioceramic compositions that further comprise a substrate, a binder, a solvent, a polymer, or an ink. In some embodiments, provided is a bioceramic composition that further comprises a substrate that comprises at least one elastomer.

In some embodiments, provided is a bioceramic composition that further comprises a polymer that is selected from the group consisting of polyoxybenzylmethylenglycolanhydride, polyvinyl chloride, polystyrene, polyethylene, polypropylene, polyacrylonitrile, polyvinyl butyral, polylactic acid, and combinations thereof. In further or additional embodiments, provided is a bioceramic composition containing an elastomer that is selected from the group consisting of poly chloroprene, nylon, a polyvinyl chloride elastomer, a polystyrene elastomer, a polyethylene elastomer, a polypropylene elastomer, a polyvinyl butyral elastomer, silicone, a thermoplastic elastomer, and combinations thereof.

In some embodiments, provided is an article containing a bioceramic composition that further comprises a substrate that comprises a material selected from the group consisting of wool, silk, cotton, canvas, jute, glass, nylon, polyester, acrylic, elastane, polychloroprene, expanded polytetrafluoroethylene-containing laminate fabrics, and combinations thereof. In still further or additional embodiments, provided is an article containing a bioceramic composition that further comprises a polygel.

For example, in one embodiment a polymeric article is prepared by mixing a bioceramic composition with the polymeric substrate, or alternatively applying the bioceramic to the substrate, while the substrate is in a liquid or fluid form. In some embodiments, the amount of bioceramic composition incorporated into the polymeric substrate or that is applied to the substrate can be any suitable amount that reflects a sufficient amount of far infrared energy. In one embodiment, the bioceramic composition is added in an amount from about 1 wt % to about 75 wt % by total weight of the article. In another embodiment, the bioceramic composition is added in an amount from about 0.01 wt % to about 25 wt % by total weight of the article. In yet another embodiment, the bioceramic composition is added in an amount from about 3 wt % to about 20 wt % by total weight of the article. In a further embodiment, the bioceramic composition is added in an amount from about 7 wt % to about 13 wt % by total weight of the article. In another embodiment, the polymeric substrate is in the form of a cloth substrate, such as a shirt, which is discussed in greater detail below.

The polymeric substrate includes any polymer that is useful for preparing an article. For example, the polymeric substrate includes at least one elastomeric polymer or at least one non-elastomeric polymer. As linked polymers and polymer systems, polymer blends that include continuous and/or dispersed phases, and the like.

Elastomers include, but are not limited to, viscoelastic polymers, such as, for example, natural rubbers, synthetic rubbers, rubbery, and rubber-like polymeric materials. One example of a synthetic rubber is polychloroprene (Neoprene). In one embodiment, the elastomer is selected from poly chloroprene, nylon, a polyvinyl chloride elastomer, a polystyrene elastomer, a polyethylene elastomer, a polypropylene elastomer, a polyvinyl butyral elastomer, silicone, a thermoplastic elastomer, and combinations thereof.

Thermoplastic elastomers (TPEs) are composite materials obtained from the combination of an elastomeric material and a thermoplastic material. TPEs are elastomeric materials that are dispersed and crosslinked in a continuous phase of a thermoplastic material. Examples of conventional TPEs include Santoprene®, available from Advanced Elastomers Systems, Inc. and Sarlink® available from DSM Elastomers, Inc.

In one embodiment, the non-elastomer is selected from a group of polymers that includes, but is not limited to, polyoxybenzylmethylenglycolanhydride, polyvinyl chloride, polystyrene, polyethylene, polypropylene, polacrylonitrile, polyvinyl butyral, polylactic acid, and the like.

With respect to an article that includes a cloth substrate and a bioceramic composition, the bioceramic composition can be applied to the cloth by any process known in the cloth/fabric art using a liquid or fluid carrier that contains the bioceramic composition. For example, a silk-screen printing process, a dot application process, a binder solution application process, a visible repeating pattern process or any other suitable method can be employed. Silk-screen printing is a printing process which uses a form—referred to as a frame or sieve—that includes a fabric with a very fine mesh, which is left permeable to the ink in the areas of the image to be reproduced and impermeable in the other areas. A dot application process uses specific devices, such as a syringe comprising a bioceramic, to apply the ceramics to particular portions of an apparel. A binder solution application process is used to dip fabrics into solutions or slurs comprising the bioceramics—in some cases this is used to impregnate the fabric with a bioceramic. A visible repeating pattern process is used to add a single pattern or repetitions of a pattern to an apparel. In one embodiment, the bioceramic composition can be incorporated into an ink, which is then silk-screened onto at least a portion of the surface of the cloth substrate.

In another embodiment, the bioceramic composition is combined with one or more liquid polymers (e.g. polyester and/or the like). The bioceramic/polymer composition is then extruded using methods known in the art to form fibers that are used in preparing a cloth substrate.

Cloth substrates useful herein include fabric or textile substrates prepared by any method known to one of skill in the cloth fabrication art. Such techniques include, but are not limited to, weaving, knitting, crocheting, felting, knotting, bonding, and the like. Suitable starting materials for the cloth substrates include natural or synthetic (e.g. polymeric) fibers and filaments. In one embodiment, the cloth substrate includes, but is not limited to, a material selected from wool, silk, cotton, canvas, jute, glass, nylon, polyester, acrylic, elastane, polychloroprene, expanded polytetrafluoroethylene-containing laminate fabrics (e.g. Gore-Tex® fabric), and combinations thereof.

With respect to an article that includes a metallic substrate, the bioceramic composition is optionally applied to the metal in a liquid/fluid form by any process known in the metal processing art. For example, the bioceramic composition is optionally incorporated into a liquid/fluid carrier, such as, but not limited to, a paint, sealant, varnish, and the like, and applied to at least a portion of the surface of the metallic substrate. The amount of bioceramic composition added to a paint or other liquid/fluid carrier can be any suitable amount. Suitable metallic substrates for use herein include any metallic substrate that is useful for preparing an article that incorporates a bioceramic composition. Exemplary metallic substrates include pure metals and alloys. In one embodiment, the metallic substrate is selected from zinc, molybdenum, cadmium, scandium, titanium, vanadium, chromium, manganese, iron, cobalt, nickel, copper, zirconium, niobium, ruthenium, rhodium, palladium, silver, tantalum, tungsten, rhenium, osmium, iridium, platinum, gold, aluminum, gallium, indium, tin, and the like.

### Apparel

Virtually any article that a bioceramic composition can be applied to or incorporated within is suitable. In one embodiment, the article is selected from apparel (e.g. garments, such as: jewelry, patches (e.g. patches that are fabricated to adhere to skin, such as transdermal patches, transdermal hydrogel patches, etc.), adhesive tape, such as kinesio, non-adhesive tape, pads, insoles, performance sleeves, uniforms, casual/leisure wear, bedding, including sheet, mattresses, covers, pillows, and pillow cases, body supports, supports, foam rollers, lotions, soaps, tape, glassware, furniture, paints, inks, labels, carpets, mats, food and/or beverage containers, drink koozies (e.g. bottle or can), headware (e.g. helmets, hats, etc.), footwear (e.g. shoes, sneakers, sandals, etc.), earphones, a surface, a sports surface, artificial grass, and the like.

In some embodiments, the apparel includes athletic apparel, sporting accessories, and sports equipment including, but not limited to, orthotic inserts, athletic shoes, uniforms, footwear, insoles, performance sleeves, diving suits, life preservers, shirts, shorts, wrist bands, arm bands, headwear (e.g. skull caps), head bands, gloves, jackets, pants, hats, and backpacks, skis, ski poles, snowboards, skateboards, in-line skates, bicycles, surf boards, water skis, jet skis, diving equipment, ropes, chains, goggles, and blankets. In some embodiments, the apparel is sporting accessories, including but not limited to blankets. In some embodiments, the apparel is configured for use in orthotic applications, including but not limited to orthotic inserts, shoes, and the like.

In another embodiment, the article is apparel selected from shirts, pants, shorts, dresses, skirts, jackets, hats, undergarments, socks, caps, gloves, scarves, diapers, and the like. In yet another embodiment, the article is jewelry selected from bracelets, necklaces, earrings, medallions, pendants, rings, and the like. In still another embodiment, the article is bedding selected from blankets, sheets, pillows, pillowcases, comforters, duvet covers, mattress covers, mattress pads, and the like. In some embodiments, the bedding is a blanket. In another embodiment, the article is a body support selected from knee wraps, elbow supports, compression arm sleeves, compression leg sleeves, wrist wraps, and the like. In some embodiments, the apparel includes casual/leisure wear.

In further or additional embodiments, provided is an article that incorporates a bioceramic composition, or an article with a bioceramic applied to it, provided that the article is selected from the group consisting of apparel, jewelry, patches, pads, insoles, bedding, body supports, foam rollers, lotions, soaps, tape, glassware, furniture, paints, inks, labels, carpets, mats, food and/or beverage containers, drink koozies, headwear, footwear, earphones, and combinations thereof. In further or additional embodiments, the article comprises apparel such as clothing. In some embodiments, the apparel is a casual/leisure wear apparel. In some embodiments, the apparel is an athletic apparel. In some embodiments, the apparel comprises a shirt, a jacket, shorts, or trousers. In still further embodiments, the apparel comprises a wrist band, a pad, a knee bracelet, an ankle bracelet, a sleeve, a performance sleeve, headwear (e.g. skull cap), a patch, footwear, or insoles.

### Cancer Patient Recovery

Treatments of cancer, including radiation and chemotherapy, are frequently associated with many severe side effects that effect the quality of life of the subject undergoing the treatment. Examples of such side effects include nausea, vomiting, loss of appetite, fatigue, fever, pain, hair loss, mouth sores, constipation, bruising, and bleeding. These side effects stemming from the treatment can cause a severe impact on the lifestyle and mental well-being of the subject in addition to any symptoms of the cancer. Thus, treatments for these side effects associated with the treatment of cancer would provide comfort to patients undergoing such cancer treatments and could improve patient outlook and compliance with cancer therapies and even improve recovery from the treatments themselves.

One aspect of the articles, compositions of matter, methods, devices, and systems described herein is a bioceramic composition that provides for improved recovery in a subject undergoing treatment for cancer. In some embodiments, the treatment for cancer comprises radiation therapy, immunotherapy, or chemotherapy. In some embodiments, the treatment for cancer comprises radiation therapy. In some embodiments, the treatment for cancer comprises immunotherapy. In some embodiments, the treatment for cancer comprises chemotherapy.

In some embodiments, the improved recovery of the subject undergoing cancer treatment is due to an effect on the mood, disposition, or feelings of well-being of the subject during treatment. In some embodiments, this effect also has the knock-on effect of enhancing the efficacy of the treatment, as the subject is more likely to continue on with the therapy. In some embodiments, the effect allows the subject to have a higher quality of life due to their increased mood, disposition, or feeling of well-being while undergoing the therapy.

In some embodiments, the subject experiences an improved quality of life, reduced fatigue, improved balance, improved hydration, an increase in total plasma antioxidants, improved quality of sleep, improved status of the autonomic nervous system, or any combination thereof after the bioceramic is applied to the body of the subject. In some embodiments, the improvement is measured as compared to the subject before the article comprising the bioceramic was applied to the body. In some embodiments, the improvement is as compared to a comparable subject who was not contacted with the bioceramic.

In some embodiments, the subject experiences an improved quality of life. In some embodiments, the improved quality of life is measured using a questionnaire. In some embodiments, the questionnaire is the World Health Organization Quality of Life Questionnaire.

In some embodiments, the subject experiences reduced fatigue. In some embodiments, the fatigue is measured by a psychometric evaluation. In some embodiments, the fatigue is measured by a fatigue pictogram psychometric evaluation.

In some embodiments, the subject experiences improved balance. In some embodiments, the improved balance is measured by baropodometry analysis.

In some embodiments, the subject experiences improved hydration. In some embodiments, the improved hydration is measured by a bioimpedance analysis. In some embodiments, the subject experiences improved body condition. In some embodiments, the improved body condition comprises a change in fat, fat free mass, lean dry mass, lean soft tissue, skeletal muscle mass, or bone mineral content.

In some embodiments, the subject experiences an increase in total plasma antioxidants.

In some embodiments, the subject experience improved sleep. In some embodiments, improved sleep is measured using a quality of sleep index (e.g., Pittsburgh Quality of Sleep Index). In some embodiments, the improved sleep comprises an improvement in one or more of subjective sleep quality, sleep latency, sleep duration, habitual sleep efficiency, sleep disturbances, use of sleep-promoting medications, daytime dysfunction, or any combination thereof. In some embodiments, the improved sleep comprises an improvement in subjective sleep quality. In some embodiments, the improved sleep comprises an improvement in subjective sleep latency. In some embodiments, the improved sleep comprises an improvement in subjective sleep duration. In some embodiments, the improved sleep comprises an improvement in subjective habitual sleep efficiency. In some embodiments, the improved sleep comprises an improvement in the number of sleep disturbances. In some embodiments, the improved sleep comprises an improvement the number of sleep-promoting medications required to be taken. In some embodiments, the improved sleep comprises a reduction in daytime dysfunction.

In some embodiments, the subject experiences an improved status of the autonomic nervous system. In some embodiments, the improved status of the autonomic nervous system comprises an improved heart rate variability (HRV). In some embodiments, the improved status of the autonomic nervous system comprises decreased markers of sympathetic activity, increased markers of parasympathetic activity, or both. In some embodiments, the improved status of the autonomic nervous system comprises decreased markers of sympathetic activity. In some embodiments, the improved status of the autonomic nervous system comprises increased markers of parasympathetic activity. In some embodiments, the improved status of the autonomic nervous system comprises decreased markers of sympathetic activity and increased markers of parasympathetic activity.

In some embodiments, the improved recovery is a mitigation of one or more side effects from the cancer therapy, such as nausea, vomiting, loss of appetite, fatigue, fever, pain, or any combination thereof. In some embodiments, the improved recovery is a mitigation of nausea from the cancer therapy. In some embodiments, the improved recovery is a mitigation of vomiting from the cancer therapy. In some embodiments, the improved recovery is a mitigation of loss of appetite from the cancer therapy. In some embodiments, the improved recovery is a mitigation of fever from the cancer therapy. In some embodiments, the improved recovery is a mitigation of pain from the cancer therapy.

In some embodiments, the improved recovery is based on: a modulation of pain, an increase in muscle endurance, an increase in stamina, an increase in muscle strength, a modulation of the cardiorespiratory system, such as an increase in respiratory capacity, an increase in flexibility, a modulation of cellular metabolism, an improvement of analgesia, an anti-oxidative effect, an anti-fibromyalgia effect, a decrease in inflammation, a decrease in oxidative stress, a modulation of cytokine levels, a modulation of blood circulation, an increase in cutaneous perfusion, a decrease in heart rate, a decrease in blood pressure, or an improvement in the quality of life.

In some embodiments, the improved recovery is observed after contacting the bioceramic or the article comprising the bioceramic for a sufficient period of time. In some embodiments, the improved recovery is observed after being in contract with the bioceramic for at least 1 hours, at least 2 hours, at least 3 hours, at least 4 hours, at least 5 hours, at least 6 hours, at least 7 hours, or at least 8 hours. In some embodiments, the improved recovery is observed after being in contract with the bioceramic for at least 1 hours, at least 2 hours, at least 3 hours, at least 4 hours, at least 5 hours, at least 6 hours, at least 7 hours, or at least 8 hours daily over a period of time. In some embodiments, the period of time is at least 2 days, at least 3 days, at least 4 days, at least 5 days, at least 6 days, at least 1 week, at least 2 weeks, at least 3 weeks, at least 4 weeks, at least 5 weeks, or at least 6 weeks. In some embodiments, the improved recovery is observed after contacting the bioceramic for a sufficient period of time (e.g., 1-8 hours) on a regular schedule, such as daily, every other daily, twice a week, three times a week, four times a week, five times a week, or six times a week.

In some embodiments, the article comprising the bioceramic is applied to the subject during sleep. In some embodiments, the article comprises bedding. In some embodiments, the bedding is selected from blankets, sheets, pillows, pillowcases, comforters, duvet covers, mattress covers, and mattress pads. In some embodiments, the bedding is a blanket. In some embodiments, the article is a shirt, a jacket, shorts or trousers. In some embodiments, the article is a shirt.

### Biomodulation Effect

Another aspect of the articles, compositions of matter, methods, devices, and systems described herein is a bioceramic composition that provides a biomodulatory or physiological effect when heated or exposed to heat, such as human radiation. In some embodiments, the biomodulatory or physiological effect comprises: a modulation of pain, an increase in muscle endurance, an increase in stamina, an increase in muscle strength, a modulation of the cardiorespiratory system, such as an increase in respiratory capacity, an increase in flexibility, a modulation of cellular metabolism, an improvement of analgesia, an anti-oxidative effect, an anti-fibromyalgia effect, a decrease in inflammation, a decrease in oxidative stress, a modulation of cytokine levels, a modulation of blood circulation, a reduction in intolerance to a cold environment, a reduction in a symptom of arthritis or vascular disease, an increase in cutaneous perfusion, a decrease in heart rate, a decrease in blood pressure, quicker recovery from injury or exercise, an esthetic effect such as a reduction in cellulite of the subject, an improvement in the quality of life.

A bioceramic composition of the disclosure has a biomodulatory or physiological effect in various subjects. In some embodiments, subjects are humans. A subject can be of any age. In some embodiments, subjects are, for example, elderly adults, adults, adolescents, pre-adolescents, children, toddlers, infants.

In some embodiments, the biomodulatory or physiological effect is a change in body composition. A body composition can be described in terms of body mass index, fat mass index, skeletal muscle mass index, percentage of body fat, or any combinations thereof. Various methods can be used to measure a body composition, such as the bioimpedance analysis. A bioimpedance analyzer can be used in a bioimpedance analysis to calculate an estimate of total body water (TBW). TBW can be used to estimate fat-free body mass and, by difference with body weight, body fat.

In some embodiments, the biomodulatory or physiological effect is an increase or a reduction in the expression level of a biomarker. Biomarkers broadly refer to any characteristics that are objectively measured and evaluated as indicators of normal biological processes, normal muscle function, pathogenic processes, or pharmacologic responses to bioceramics. Unless otherwise noted, the term biomarker as used herein specifically refers to biomarkers that have biophysical properties, which allow their measurements in biological samples (e.g., saliva, plasma, serum, cerebrospinal fluid, bronchoalveolar lavage, biopsy).

Examples of biomarkers include nucleic acid biomarkers (e.g., oligonucleotides or polynucleotides), peptides or protein biomarkers, cytokines, hormones, or lipids. In some embodiments, an article comprising a bioceramic composition of the disclosure has a biomodulatory or physiological effect on a biomarker.

In some embodiments, a biomarker is a cytokine. Non-limiting examples of cytokines include: a) cytokines in the IL-2 subfamily, for example erythropoietin (EPO) and thrombopoietin (TPO); b) the interferon (IFN) subfamily, for example IFN-γ; c) the IL-6 subfamily; d) the IL-10 subfamily; e) the IL-1 subfamily, for example, IL-1 and IL-18, f) IL-17; or g) tumor necrosis factor family, for example tumor necrosis factor alpha (TNF-alpha or TNF-α). In some embodiments, an article comprising a bioceramic composition of the disclosure has a biomodulatory or physiological effect on a cytokine. In some embodiments, the cytokine is associated with inflammation, pain, muscle endurance, a modulation of the cardiorespiratory system, a modulation of cellular metabolism, analgesia, cellular oxidation, fibromyalgia effect, or another condition described herein.

In some embodiments, a biomarker is a wild-type protein or a protein that has been modified from a native state. For example, protein carbonylation is a type of protein oxidation that can be promoted by reactive oxygen species. It usually refers to a process that forms reactive ketones or aldehydes that are amenable to reacting with 2,4-dinitrophenylhydrazine (DNPH) to form hydrazones. Direct oxidation of side chains of lysine, arginine, proline, and threonine residues, among other amino acids, in the “primary protein carbonylation” reaction produces DNPH detectable protein products. In some embodiments, an article comprising a bioceramic composition of the disclosure has a biomodulatory or physiological effect on a protein. In some embodiments, the protein is associated with inflammation, pain, muscle endurance, a modulation of the cardiorespiratory system, a modulation of cellular metabolism, analgesia, cellular oxidation, fibromyalgia effect, or another condition described herein.

In some embodiments, a biomarker is a wild-type lipid or a lipid that has been modified from a native state. For example, lipid peroxidation refers to the oxidative degradation of lipids. It is the process in which free radicals remove electrons from the lipids in cell membranes, resulting in cell damage. In some embodiments, an article comprising a bioceramic composition of the disclosure has a biomodulatory or physiological effect on a lipid. In some embodiments, the lipid is associated with inflammation, pain, muscle endurance, a modulation of the cardiorespiratory system, a modulation of cellular metabolism, analgesia, cellular oxidation, fibromyalgia effect, or another condition described herein.

In some embodiments, the bioceramic composition provides a biomodulatory or physiological effect that comprises a change that is statistically significant. In further or additional embodiments, the biomodulatory or physiological effect comprises a change that is at least 5% in the effect. In some embodiments, the biomodulatory or physiological effect comprises a change that is at least 10% in the effect. In still further or additional embodiments, the biomodulatory or physiological effect is pain relief, and the pain is caused by a physical activity. In still further or additional embodiments, the biomodulatory or physiological effect is inflammation.

The time needed for a bioceramic of the disclosure to modulate the effect of a biomarker often depends on the prevalent quantity, distribution and concentration of the bioceramic in contact with the subject. In some embodiments, a biomodulatory or physiological effect of a bioceramic of the disclosure is achieved within less than 10 minutes, less than 1 hour, less than 6 hours, less than 12 hours, less than 24 hours, less than 48 hours, less than 72 hours, less than 1 week, less than 2 weeks, less than 3 weeks, less than 4 weeks, less than 2 months, less than 6 months, or less than 12 months of a use of an apparel comprising a bioceramic.

### Non-Invasive Methods of Providing Biomodulation to a Subject

Another aspect of the subject matter described herein is a non-invasive method of providing a biomodulatory or physiological effect in or to a subject comprising contacting an article comprising a bioceramic to the skin of the subject, provided that when heated or exposed to heat, the bioceramic composition provides far infrared thermal radiation and a biomodulatory or physiological effect to the subject in a non-invasive manner.

For example, in some embodiments, provided is a bioceramic composition that when heated or exposed to heat provides a biomodulatory or physiological effect when the article is applied to a subject, comprising:
a. about 20 wt % to about 80 wt % kaolinite (Al₂Si₂O₅(OH)₄);
b. about 1 wt % to about 30 wt % tourmaline;
c. about 1 wt % to about 40 wt % aluminum oxide (Al₂O₃);
d. about 1 wt % to about 40 wt % silicon dioxide (SiO₂); and
e. about 1 wt % to about 20 wt % one additional oxide; provided that the amounts are by total weight of the bioceramic composition. In some embodiments, the one additional oxide is selected from the group consisting of titanium dioxide (TiO₂), magnesium oxide (MgO), and zirconium dioxide (ZrO₂). In some embodiments, the one additional oxide is TiO₂.

In further or additional embodiments, provided is a bioceramic composition of matter that when heated or exposed to heat provides a biomodulatory or physiological effect when the article is applied to a subject, comprising:
a. about 40 wt % to about 60 wt % kaolinite (Al₂Si₂O₅(OH)₄);
b. about 5 wt % to about 15 wt % tourmaline;
c. about 15 wt % to about 25 wt % aluminum oxide (Al₂O₃);
d. about 10 wt % to about 20 wt % silicon dioxide (SiO₂); and
e. about 1 wt % to about 20 wt % one additional oxide; provided that the amounts are by total weight of the bioceramic composition. In some embodiments, the bioceramic composition comprises kaolinite in a range from about 45 wt % to about 55 wt %. In further or additional embodiments, provided is a bioceramic composition that comprises kaolinite in the range from about 47 wt % to about 53 wt %. In further or additional embodiments, provided is a bioceramic composition that contains kaolinite in a range from about 48 wt % to about 52 wt %. In some embodiments, the bioceramic comprises about 7 wt % to about 13 wt % tourmaline. In some embodiments, the bioceramic comprises about 8 wt % to about 12 wt % tourmaline. In some embodiments, the bioceramic comprises about 17 wt % to about 23 wt % aluminum oxide. In some embodiments, the bioceramic comprises about 18 wt % to about 22 wt % aluminum oxide. In some embodiments, the bioceramic comprises about 12 wt % to about 18 wt % silicon dioxide. In some embodiments, the bioceramic comprises about 13 wt % to about 17 wt % silicon dioxide. In some embodiments, the bioceramic comprises about 5 wt % to about 15 wt % of the additional oxide. In some embodiments, the bioceramic comprises about 5 wt % to about 12 wt % of the additional oxide. In some embodiments, the bioceramic comprises about 5 wt % to about 10 wt % of the additional oxide. In some embodiments, the additional oxide is a metal oxide. In some embodiments, the one additional oxide is selected from the group consisting of titanium dioxide (TiO₂), magnesium oxide (MgO), and zirconium dioxide (ZrO₂). In some embodiments, the one additional oxide is TiO₂.

In some embodiments, provided is a bioceramic composition that comprises
a. about 50 wt % kaolinite (Al₂Si₂O₅(OH)₄);
b. about 10 wt % tourmaline;
c. about 18 wt % aluminum oxide (Al₂O₃);
d. about 14 wt % silicon dioxide (SiO₂); and
e. about 8 wt % one additional oxide. In some embodiments, the one additional oxide is selected from the group consisting of titanium dioxide (TiO₂), magnesium oxide (MgO), and zirconium dioxide (ZrO₂). In some embodiments, the one additional oxide is TiO₂.

In some embodiments, the biomodulatory or physiological effect comprises: a modulation of pain, an increase in muscle endurance, a modulation of the cardiorespiratory system, a modulation of cellular metabolism, analgesia, an anti-oxidative effect, an anti-fibromyalgia effect, a decrease in inflammation, a decrease in oxidative stress, a decrease in endoplasmic reticulum stress, a modulation of cytokine levels, a modulation of blood circulation, a reduction in intolerance to a cold environment, a reduction in a symptom of arthritis or vascular disease, an increase in cutaneous perfusion, a decrease in heart rate, a decrease in blood pressure, an esthetic effect, such as reduction of body measurements, reduction of weight, or a reduction in cellulite of the subject.

In some embodiments, the bioceramic composition provides a biomodulatory or physiological effect that comprises a change that is statistically significant. In further or additional embodiments, the biomodulatory or physiological effect comprises a change that is at least 5% in the effect. In some embodiments,

In some embodiments, provided is an article that incorporates a bioceramic composition, or an article with a bioceramic applied to it, provided that the article is selected from the group consisting of apparel, jewelry, patches, pads, insoles, bedding, body supports, foam rollers, lotions, soaps, tape, glassware, furniture, paints, inks, labels, carpets, mats, food and/or beverage containers, drink koozies, headwear, footwear, earphones, and combinations thereof. In further or additional embodiments, the article comprises apparel such as clothing. In some embodiments, the apparel comprises a shirt, a jacket, shorts or trousers. Ion still further embodiments, the apparel comprises a wrist band, a pad, a knee bracelet, an ankle bracelet, a sleeve, or a patch. In some embodiments, the article comprises a surface, a sports surface, or artificial grass.

A bioceramic composition of the invention can be a combination of any compounds described herein with other chemical components, such as carriers, stabilizers, diluents, dispersing agents, suspending agents, thickening agents, and/or excipients. The bioceramic can be administered directly or indirectly to the skin of a subject. In some cases, the active compounds can be applied to an article and exposed to a subject indirectly. In other cases, the active compounds can be applied directly to the skin of a subject.

### Patterns

A bioceramic may be added to an article of apparel in a variety of regular or irregular patterns. A bioceramic pattern may cover the entirety of the surface of an apparel or a pattern may cover a portion of an apparel. A bioceramic pattern covering an apparel may have regions of discontinuity having a variety of shapes and sizes. For example, a pattern may be a honeycomb pattern (e.g., with hexagonal regions of discontinuity), a grid pattern (e.g., with square-shaped or rectangular regions of discontinuity), a random pattern (e.g., with regions of discontinuity distributed randomly), and so forth. In general, the regions of discontinuity may be distributed across the surface at intervals that are regularly spaced or not regularly spaced. The regions of discontinuity may be formed with a variety of regular or irregular shapes such as, for example, circular, half-circular, diamond-shaped, hexagonal, multi-lobal, octagonal, oval, pentagonal, rectangular, square-shaped, star-shaped, trapezoidal, triangular, wedge-shaped, and so forth. If desired, one or more regions of discontinuity may be shaped as logos, letters, or numbers. In some embodiments, the regions of discontinuity may have sizes of about 0.1 mm, about 1 mm, about 2 mm, about 3 mm, about 4 mm, about 5 mm, about 6 mm, about 7 mm, about 8 mm, about 9 mm, about 10 mm, or other desired distance. In some embodiments, the regions of discontinuity may range from 0.1 mm to about 1 mm, from 1 mm to about 5 mm, from 1 mm to about 10 mm, from 1 mm to about 15 mm, from 1 mm to about 20 mm, from 1 mm to about 25 mm, from 1 mm to about 30 mm, or other desired distance. In general, the regions of discontinuity may have the same or different shapes or sizes.

A bioceramic pattern may be applied as a coat covering an interior and/or an exterior surface of an article of apparel. A bioceramic pattern may permeate a material, such as a fabric. A bioceramic pattern may cover various portions of a fabric in a continuous, discontinuous, regular, or irregular pattern, or any combination thereof. A bioceramic pattern may permeate less than 1%, less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, less than 85%, less than 90%, less than 95%, or less than 99%, of an interior surface of an article of apparel, an exterior surface of an article of apparel, or any combination thereof.

The following non-limiting examples serves to further illustrate the present invention.

### EXAMPLES

### Example 1: Preparation of a Bioceramic Powder Composition

The kaolinite is extracted in the outskirts of the city of Parintins, in the Amazon State, Brazil. The city is located in the Lower Amazon Region (coordinates: latitude: 2° 37′ 42″ south/longitude: 560 44′ 11″ west of Greenwich, 50 m above sea level). Alternatively, the kaolinite is obtained by purchasing it from a mining company/supplier.

The extracted kaolinite is washed with hydrogen peroxide (H₂O₂) and allowed to dry. The dried kaolinite is then finely ground and mixed with tourmaline; aluminum oxide (Al₂O₃); silicon dioxide (SiO₂); and zirconium oxide (ZrO₂) until a homogeneous mixture is achieved. The resulting bioceramic composition contains 50 wt % kaolinite, 10 wt % tourmaline, 18 wt % aluminum oxide, 14 wt % silicon dioxide, and 8 wt % titanium oxide.

A bioceramic composition is also synthesized. The resulting bioceramic contains any composition described herein, including about 50% kaolinite, about 10% tourmaline, about 18% aluminum oxide, about 14% silicon dioxide, and about 8% titanium oxide.

### Example 2: Application to Clothing

A bioceramic of the disclosure is a refractory, inorganic, polycrystalline composition that can be reduced to powdered format by grinding, crushing, or another suitable. In powder form, a bioceramic is incorporated into a range of materials; including various types of polymers and inks. A powered bioceramic is incorporated into a cloth substrate to applying an ink comprising the bioceramic to the cloth.

A cloth substrate that includes 88 wt % polyamide and 12 wt % elastane was obtained. A bioceramic composition prepared according to the method of Example 1 was incorporated onto a plastisol ink in an amount of 10-50 wt % and mixed. The mixture was applied to the cloth substrate using a traditional silkscreen process. The specific type of ink was selected based on the chosen fabric.

### Example 3: Silkscreen Application of Bioceramics to Clothing (e.g., a Shirt)

Concentration: ceramic materials are mixed with the ink at a 30% concentration of the total weight/volume.

Mixing process: ceramics were added to the ink gradually. Regular mixing process was applied using a mixer that is customarily used for pigment and ink mixing. The materials are mixed until a consistent and uniform mix/slurry was achieved. The process was fast as the ceramics mix well with all different types of inks.

Durability of the slurry/mix: a well-sealed mixture is stored and used up to one week after production.

Application: the bioceramic material was applied in the same manner as regular ink through a silk-screening process. It was observed that due to their particle size, the ceramic materials may scratch the screens. It is recommended that after every 1000 shirts the screens are checked/inspected and if needed they should be replaced, especially to avoid defects on the application and the look of the logo.

Fabric selection: the ceramics did not cause any observable damage to the fabric. It was observed that fabrics that are too porous or cannot go through the regular drying process commonly used in silk-screening should be avoided.

Ink selection: the ceramics may increase ink density and the type of ink should be selected by a person of ordinary skill in the art based on the type of fabric used.

Drying process after silk-screening: due to the fact that the ceramics may contain a small amount of moisture, it was observed that the drying may take longer than usual. The duration and intensity of the process depend upon the type of fabric and ink selected. After the first experimental run with the fabric and ink selected, the product should be subjected to a wash test to make sure the ink does not come off or crack.

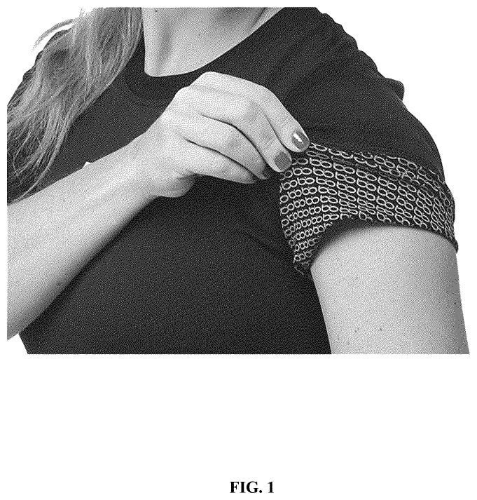

A silk screening process is used to provide a ceramic to an apparel with a desired pattern. A silk screen approach is used to intercalate an imprint with a pattern into a shirt. FIG. 1 illustrates a shirt comprising a ceramic composition of the disclosure that was fabricated with a silkscreen application.

### Example 4: Dot Application Approach of Applying Bioceramics to Clothing (e.g., a Shirt)

Concentration: ceramic materials are mixed with either silicone or a polymer, such as m-gel at a ratio of 1 part ceramic to 9 parts silicone. Alternatively, ceramic materials are mixed with a polymer, such as m-gel at a ratio of 1 part ceramic to 9 parts m-gel.

Application: a device has been used to apply the ceramic dots to the fabric with a desired pattern. A dot application approach is used to, for instance, intercalate a pattern into a shirt. FIG. 1 illustrates a shirt comprising a ceramic composition of the disclosure.

Fabric selection: the ceramics do not cause any observable damage to the fabric. A dot application approach is used to apply a bioceramic to specific areas of fabrics or apparel. In some instances, a dot application approach is used to apply the ceramics to specific areas of the piece even on top of the silkscreen in order to achieve a higher concentration of ceramics per surface area. A dot application approach is used around the shoulders, elbows, or any area where it is desirable to apply a higher concentration of ceramics.

### Example 5: Binder/Solution Approach of Applying Bioceramics to Clothing (e.g., a Shirt)

Concentration: as an alternative to mixing a ceramic to an ink and using a silkscreen or dot approach to apply the ceramic to a fabric, a binder solution approach is used. A binder solution comprises up to 50% ceramic and up to 50% binder solution or slurry.

Application: a fabric is placed in a slurry solution comprising a ceramic and a binder at a desired concentration. The fabric is removed from the slurry solution and allowed to dry. The fabric is now impregnated or infused with a ceramic of the disclosure. The fabric that is impregnated or infused with the ceramic is directly placed in contact with a skin of a subject.

### Example 6: Visible Repeating Pattern Approach of Applying Bioceramics to Clothing (e.g., a Shirt)

Concentration: a first solution comprising an ink is prepared. A second solution, slur, or binder comprising from about 10% ceramic to about 50% ceramic is prepared by mixing a ceramic of the disclosure with the ink, slur, or binder.

Application: a first pattern is sprayed, printed, silk screened, or otherwise applied to a fabric. The first pattern consists of ink and does not contain a bioceramic material. A second pattern comprising from about 10% ceramic to about 50% ceramic is subsequently sprayed, printed silk screened, or otherwise applied to the surface of the first pattern. Optionally, a silicone coating is applied over the second pattern to provide a glossy appearance of the pattern applied to the fabric. Optionally, a silicone coating is mixed with a concentration of the ceramics prior to being applied as a coat.

### Example 7: Fabrication of a Pad

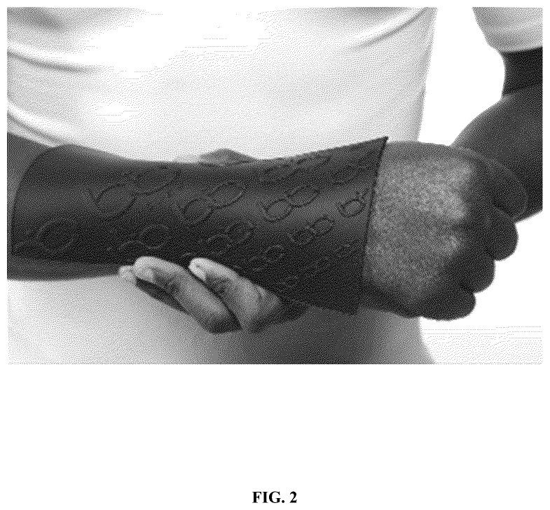

A thermoplastic elastomer (TPE) is liquefied with a bioceramic of the disclosure. The TPE and the ceramic are mixed at a concentration of about 1 part ceramic to 1 part TPE, 1 part ceramic to 2 parts TPE, 1 part ceramic to 3 parts TPE, 1 part ceramic to 4 parts TPE, 1 part ceramic to 5 parts TPE, 1 part ceramic to 6 parts TPE, 1 part ceramic to 7 parts TPE, 1 part ceramic to 8 parts TPE, or 1 part ceramic to 9 parts TPE. The liquefied mix is placed on a mold. The mix of TPE and bioceramic is allowed to solidify to provide an apparel with the shape of the mold. The apparel is removed from the mold. The apparel is a thermoplastic pad comprising a bioceramic, such as the pad illustrated in FIG. 2.

### Example 8: Infrared Transmittance of Bioceramics

Objective: to compare the infrared transmittance of a bioceramic of the instant disclosure (comprising 18% aluminium oxide, 14% silicon dioxide, 50% kaolinite, 8% zirconium oxide, and 10% tourmaline) to a distinct bioceramic composition (comprising 20% aluminum, 3% titanium, 11% magnesium oxide, 6% diiron trioxide, and 60% silica).

Methods: the infrared transmittance of powdered samples (particle size=about 25 micrometers) of the bioceramic powders was taken using a Bruker spectrometer (Model Spectrum VERTEX 70, OPUS 6.5 software). Transmittance (%) ratings were determined with a resolution of 4 cm-1 and 72 scans at a scan range from 350 cm-1 to 4000 cm-1.

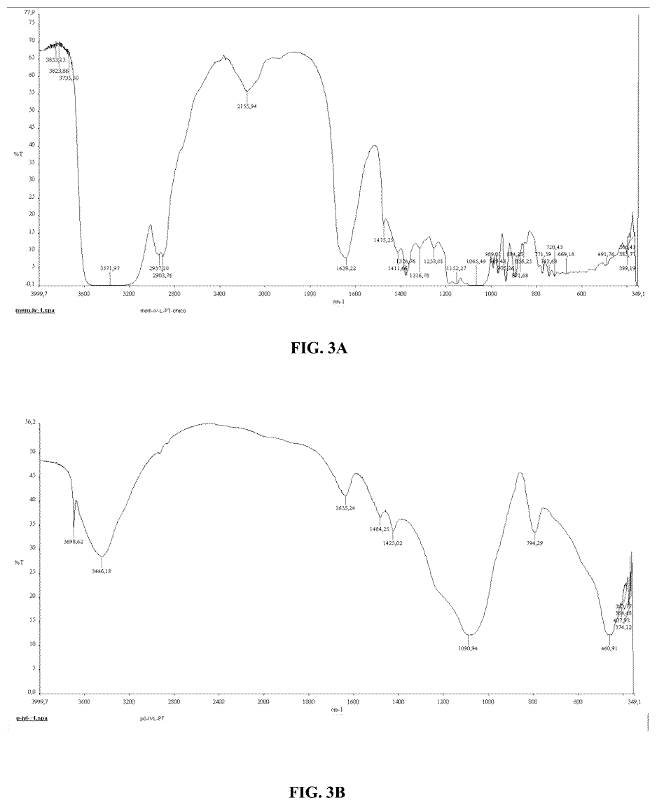

FIG. 3A illustrates the infrared transmittance of a bioceramic composition of the instant disclosure comprising 18% Aluminium oxide, 14% silicon dioxide, 50% kaolinite, 8% zirconium oxide, and 10% tourmaline. FIG. 3B illustrates the infrared transmittance of a bioceramic composition comprising 20% aluminum, 3% titanium, 11% magnesium oxide, 6% diiron trioxide, and 60% silica

### Example 9: Evaluation of Bioceramics in Cancer Patients Undergoing Radiation or Chemotherapy

A study was conducted to evaluate the effects of far-infrared emitting ceramics (cFIR) apparel in cancer patients after receiving chemotherapy or radiotherapy. The study was a randomized, double-blinded, placebo-controlled trial. The study investigated the following outcomes: Quality of Life (questionnaire); Fatigue (questionnaire); Balance (baropodometry); Hydration and Body Composition (bioimpedance); Total Antioxidants in Plasma (Biochemical assessment); Quality of Sleep (questionnaire); and Status of Autonomic Nervous System (Heart Rate Variability—HRV).

Volunteers were screened through an online questionnaire. Screened volunteers signed an Informed Consent Form with detailed research protocol and study expectations. The following screening evaluations were conducted during the screening process: Health History Questionnaire; and Anthropometrics (Height, Weight, and BMI). Twenty participants were selected with no ex or age restrictions. The selected participants were randomly divided by single draw into two experimental groups of ten per group. Group A (n=10) were asked to wear a bioceramic imprinted shirt during the night time (sleep) for the duration of the study (6 weeks). The bioceramic imprinted on the shirt was a 50 wt % kaolinite, 10 wt % tourmaline; 18 wt % aluminum oxide; 14 wt % silicon dioxide; and 8 wt % titanium oxide bioceramic. Group B was asked to wear a placebo implanted shirt for the same duration.

Evaluations were conducted at baseline and after 6 weeks of study. The following evaluations were performed: 1) Quality of Life (Quality of Life (WHOQOL-BREF—World Health Organization Quality of Life Questionnaire); 2) Fatigue (Fatigue pictogram—Psychometric evaluation. de Faria Mota et al., 2009, Rev Esc Enferm USP; 43(Spe):1079-86); 3) Balance (Baropodometry Platform, Medicapteurs, France); 4) Hydration and Body Composition (Bioimpedance Analysis—RJL Quantum IV Bod Composition Analyzer; RJL Systems, USA); 5) Total Antioxidants in Plasma (Serum Biochemical Assessment—Spectrophotometry); 6) Quality of Sleep (Pittsburgh Quality of Sleep Index); and 7) Status of the Autonomic Nervous System (Heart Rate Variability (HRV) analysis, Nerve Express, USA).

The resulting data was subjected to the following statistical analysis: 1) Identification of Outliers by the ROUT method (Prism, version 8.0—La Jolla, California, USA); 2) Normality tests to determine if data set were well-modeled by a normal distribution (Anderson-Darling test, D'Agostino & Pearson test, Shapiro-Wilk test & Kolmogorov-Smirnov test); 3) For parametric data sets: Unpaired t test, 2-tailed with a 95% confidence interval & One-Way Anova followed by Tukey multiple comparison tests; 4) For non-parametric data sets: Mann-Whitney test 2-tailed with a 95% confidence interval & One-Way Anova followed by Dunns multiple comparison tests. Unless otherwise specified, data are presented as ±standard deviation. Values of P<0.05 were considered statistically significant.

The intervention group included 6 females and 4 males with an average age of 55.6, an average height of 1.68 meters, an average weight of 75.3 kg, and an average body mass index of 26.67. The placebo group included 7 females and 3 males with an average age of 53.40, an average height of 1.6 meters, an average weight of 61.4 kg, and an average body mass index of 23.77.

### Results

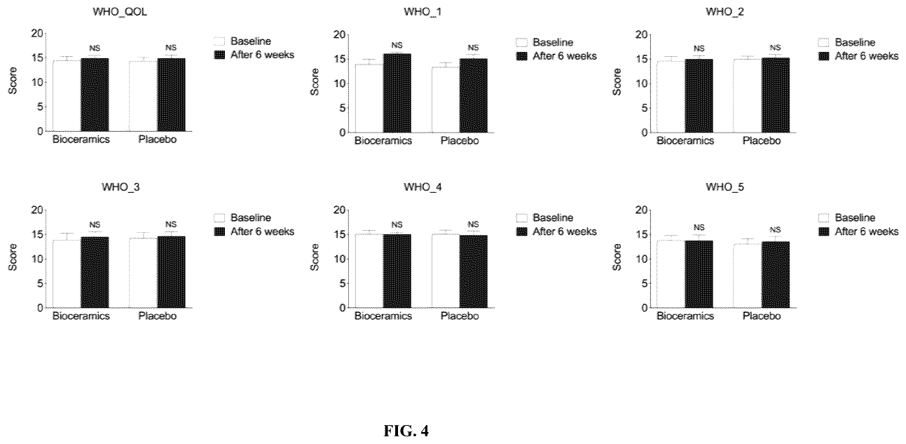

Quality of Life—The results from the Quality of Life surveys (WHOQOL-BREF—World Health Organization Quality of Life Questionnaire) taken by the patients at the start and completion of the study are shown in FIG. 4. Each point represents the mean of the values obtained with 10 participants (+1), and the vertical lines indicate the SD. WHO_QOL: Total score; WHO_1: physical health domain; WHO 2: psychological domain; WHO_3: social relationships domain; WHO_4: environmental domain; WHO_5: overall quality of life and general health; NS: Non-significant.

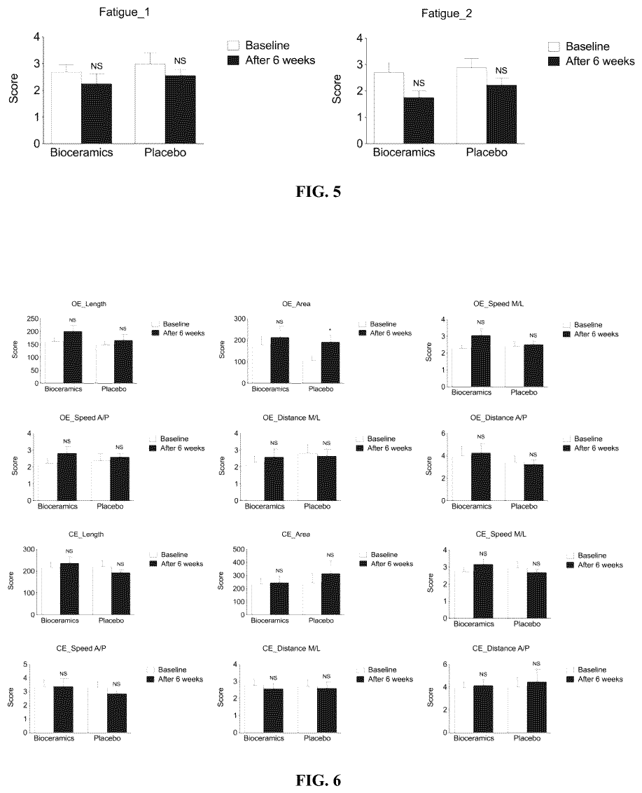

Fatigue—The results from the fatigue pictogram psychometric evaluation (de Faria Mota et al., 2009, Rev Esc Enferm USP; 43(Spe):1079-86) performed on the patients at the start and completion of the study are shown in FIG. 5. Each point represents the mean of the values obtained with 10 participants (+1), and the vertical lines indicate the SD. Fatigue_1: Fatigue Intensity; Fatigue 2: Impact of Fatigue; NS: Non-significant.

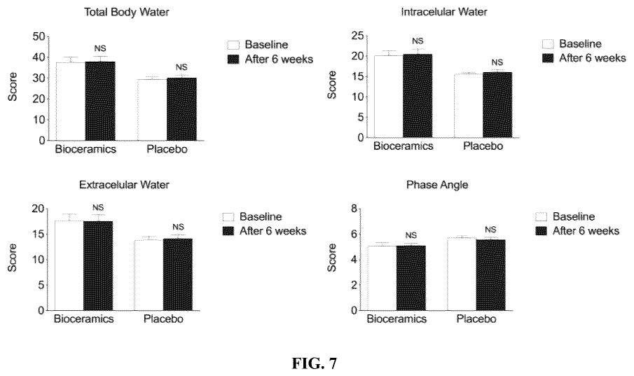

Balance—The results from the balance baropodometry platform evaluation performed on the patients at the start and completion of the study are shown in FIG. 6. Each point represents the mean of the values obtained with 10 participants (1), and the vertical lines indicate the SD. CE: Closed Eyes Assessment; OE: Open Eyes Assessment; A/P: Anteroposterior Plane; M/L: Mediolateral Plane; NS: Non-significant; The asterisk (*) denotes the significance level (*P<0.05).

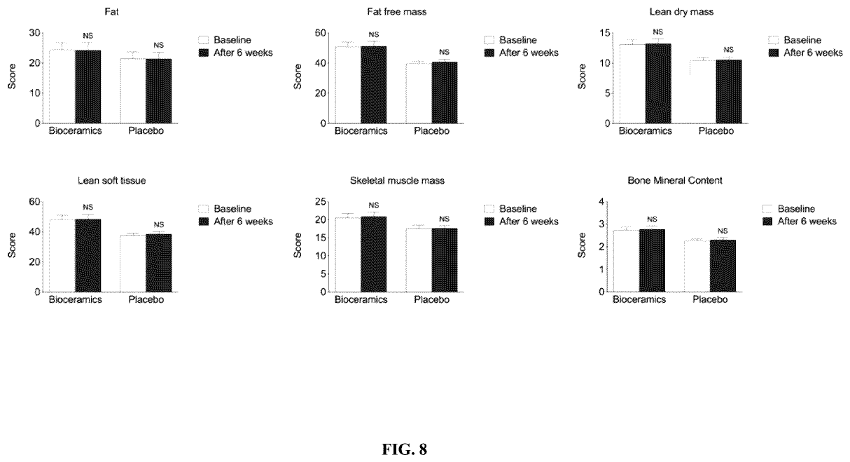

Hydration—The results from the hydration bioimpedance analysis (RJL Quantum IV Body Composition Analyzer; RJL Systems, USA) evaluation performed on the patients at the start and completion of the study are shown in FIG. 7 and FIG. 8.

In FIG. 7, each point represents the mean of the values obtained with 10 participants (±1), and the vertical lines indicate the SD. Total Body Water (TBW): is all of the water throughout the body, both inside and outside of the cells; Intra-Cellular Water (ICW): represents the amount of water inside cells; Extra-Cellular Water (ECW): represents the amount of water outside of the cells; Phase Angle (PA): PA reflects the relative contributions of fluid (resistance), and cellular membranes (capacitive reactive). It is calculated as the arc-tangent of Reactance over resistance, measured in degrees; NS: Non-significant.

In FIG. 8, each point represents the mean of the values obtained with 10 participants (±1), and the vertical lines indicate the SD. Fat: Overall Fat; Fat Free Mass (FFM): also called Lean Body Mass, is everything in the body, except Fat; Lean Dry Mass (LDM): is what is left after subtracting all of the water from Fat Free Mass; Lean Soft Tissue (LST): In the same way that LDM is the result of removing all water from Fat-Free Mass. Lean Soft Tissue is the result of subtracting Bone Mineral Content from Fat-Free Mass. This includes organs, muscles, connective and supportive tissues, as well as all of Total Body Water; Skeletal Muscle Mass (SMM): SMM are the muscles responsible for posture and movement; Bone Mineral Content (BMC): Bones are dynamic organs that include cells, blood vessels, collagen and mineral deposits. BMC is only an estimate of the minerals in the bones and does not represent the total weight of the skeleton. It is part of Fat-Free Mass; NS: Nonsignificant.

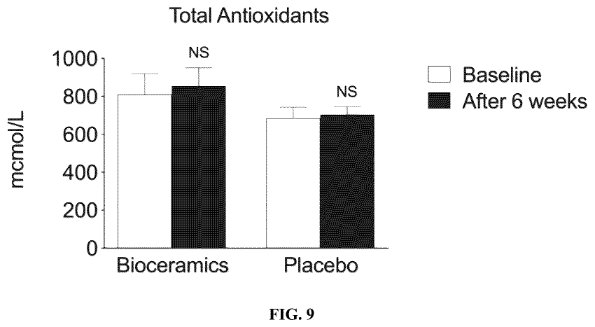

Total Plasma Antioxidants—The results from the plasma antioxidant measurements (Serum Biochemical Assessment—Spectrophotometry) performed on the patients at the start and completion of the study are shown in FIG. 9. Each point represents the mean of the values obtained with 10 participants (+1), and the vertical lines indicate the SD. NS: Nonsignificant.

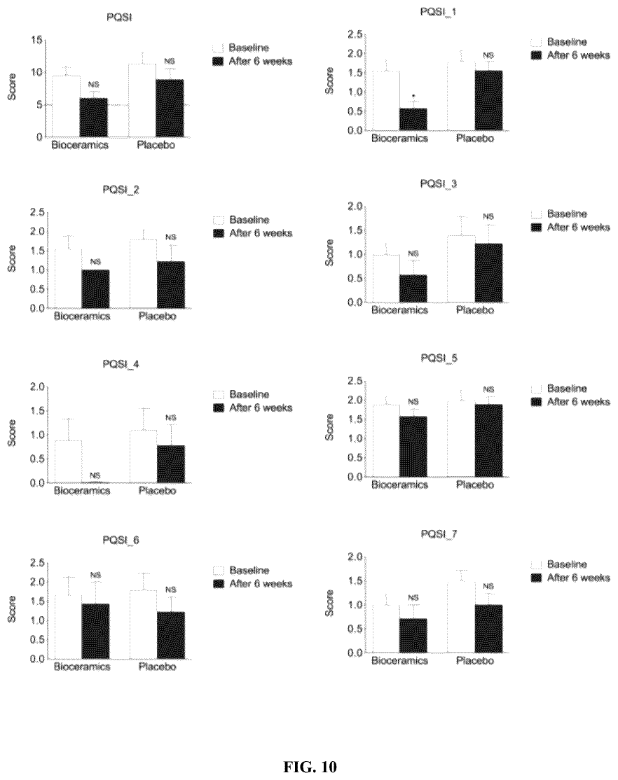

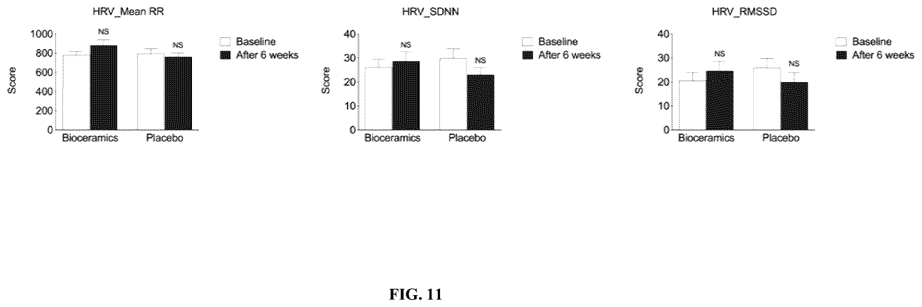

Quality of Sleep—The results from the quality of sleep analysis (Pittsburgh Quality of Sleep Index) performed on the patients at the start and completion of the study are shown in FIG. 10. Each point represents the mean of the values obtained with 10 participants (±1), and the vertical lines indicate the SD. PQSI: Pittsburgh Quality of Sleep Index Total Score; PQSI_1: Component 1: subjective sleep quality; PQSI_2: Component 2: sleep latency; PQSI_3: Component 3: sleep duration; PQSI 4: Component 4: habitual sleep efficiency; PQSI_5: Component 5: sleep disturbances; PQSI_6: Component 6: use of sleep-promoting medications; PQSI_7: Component 7: daytime dysfunction; NS: Non-significant; The asterisk (*) denotes the significance level (*P<0.05).

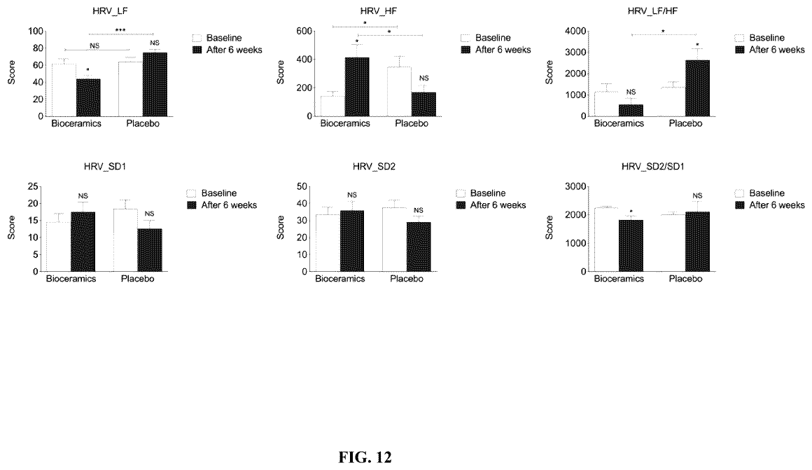

Status of Autonomic Nervous System—The results from the Heart Rate Variability (HRV) analysis (Nerve Express, USA) performed on the patients at the start and completion of the study are shown in FIG. 11 and FIG. 12. Statistically significant results were detected when comparing Baseline and post-intervention in the Bioceramics group in the PQSI Component 1: subjective sleep quality. Results suggest that cFIR imprinted shirts positively affect self-assessed quality of sleep. Statistically significant results were detected when comparing Baseline and post-intervention in the Bioceramics group in the following parameters: HRV_LF: Low Frequency Band: general indicator of aggregate modulation of both the sympathetic and parasympathetic branches of the Autonomic Nervous System; HRV_HF: High Frequency Band: index of modulation of the parasympathetic branch of the autonomic nervous system; HRV_LF/HF: Ratio of LF-to-HF power: a low LF/HF ratio reflects parasympathetic dominance. This is seen when we conserve energy and engage in tend-and-befriend behaviors. In contrast, a high LF/HF ratio indicates sympathetic dominance, which occurs when we engage in fight-or-flight behaviors or parasympathetic withdrawal. Perfect balance is ZERO (0); and HRV_SD2/SD1: Ratio of SD1-to-SD2. The ratio of SD1/SD2, which measures the unpredictability of the RR time series, is used to measure autonomic balance when the monitoring period is sufficiently long and there is sympathetic activation. SD1/SD2 is correlated with the LF/HF ratio. Perfect balance is ZERO (0). Results suggest that cFIR imprinted shirts positively affect the Status of the Autonomic Nervous System, decreasing markers of sympathetic activity, increasing markers of parasympathetic activity and bringing the overall Autonomic Nervous System to a more “balanced” state.

FIG. 11 shows Status of the Autonomic Nervous System (Heart Rate Variability—HRV). Each point represents the mean of the values obtained with 10 participants (±1), and the vertical lines indicate the SD. HRV_Mean RR: Mean RR: intervals between successive heartbeats (where R is a point corresponding to the peak of the QRS complex of the ECG wave; and RR is the interval between successive Rs); HRV_SDNN: Standard deviation of NN intervals; HRV_RMSSD: RR normal-to-normal intervals; a marker of overall HRV activity; NS: Non-significant. (Reference: Shaffer & Ginsberg. Front Public Health. 2017).

FIG. 12 shows Status of the Autonomic Nervous System (Heart Rate Variability—HRV). Each point represents the mean of the values obtained with 10 participants (±1), and the vertical lines indicate the SD. HRV_LF: Low Frequency Band: general indicator of aggregate modulation of both the sympathetic and parasympathetic branches of the Autonomic Nervous System; HRV_HF: High Frequency Band: index of modulation of the parasympathetic branch of the autonomic nervous system; HRV_LF/HF: Ratio of LF-to-HF power. a low LF/HF ratio reflects parasympathetic dominance. This is seen when we conserve energy and engage in tend-and-befriend behaviors. In contrast, a high LF/HF ratio indicates sympathetic dominance, which occurs when we engage in fight-or-flight behaviors or parasympathetic withdrawal. Perfect balance is ZERO (0); HRV_SD1: Poincare plot standard deviation perpendicular the line of identity; HRV_SD2: Poincare plot standard deviation along the line of identity; HRV_SD2/SD1: Ratio of SD1-to-SD2. The ratio of SD1/SD2, which measures the unpredictability of the RR time series, is used to measure autonomic balance when the monitoring period is sufficiently long and there is sympathetic activation. SD1/SD2 is correlated with the LF/HF ratio. Perfect balance is ZERO (0); NS: Non-significant; The asterisk (*) denotes the significance level (*P<0.05). (Reference: Shaffer & Ginsberg. Front Public Health. 2017).

Results suggest that cFIR imprinted shirts positively affect self-assessed quality of sleep & the status of the Autonomic Nervous System, decreasing markers of sympathetic activity, increasing markers of parasympathetic activity and bringing the overall Autonomic Nervous System to a more “balanced” state. Combined these results may indicate positive effects of far-infrared emitting ceramics (cFIR) apparel in cancer patients after Chemotherapy or Radiotherapy.

We claim:

1. A method of improving recovery in a subject who has undergone and/or is undergoing treatment for cancer, comprising contacting an article comprising a bioceramic with the subject's body, wherein when heated or exposed to heat, the bioceramic composition provides far infrared thermal radiation to the subject, thereby providing an improvement in recovery in the subject.

2. The method of claim 1, wherein the treatment for cancer comprises radiation therapy, chemotherapy, immunotherapy, or any combination thereof.

3. The method of claim 1 or 2, wherein the treatment for cancer comprises chemotherapy.

4. The method of any one of claims 1-3, wherein the article comprising the bioceramic contacts skin of the subject.

5. The method of any one of claims 1-4, wherein the improvement comprises an improved quality of life, reduced fatigue, improved balance, improved hydration, an increase in total plasma antioxidants, improved quality of sleep, improved status of the autonomic nervous system, or any combination thereof.

6. The method of any one of claims 1-5, wherein the improvement comprises improved quality of life as measured using the World Health Organization Quality of Life Questionnaire.

7. The method of claim 5 or 6, wherein the improvement comprises reduced fatigue as measured by a fatigue pictogram psychometric evaluation.

8. The method of any one of claims 1-7, wherein the improvement comprises improved balance as measured by baropodometry analysis.

9. The method of any one of claims 1-8, wherein the improvement comprises improved hydration as measured by a bioimpedance analysis.

10. The method of any one of claims 1-9, wherein the improvement comprises an increase in total plasma antioxidants.

11. The method of any one of claims 1-10, wherein the improvement comprises improved quality of sleep as measured by the Pittsburgh Quality of Sleep Index.

12. The method of any one of claims 1-11, wherein the improvement comprises improved status of the autonomic nervous system.

13. The method of any one of claims 1-12, wherein the improvement comprises a reduced heart rate variability (HRV).

14. The method of any one of claims 1-13, wherein the improved status of the autonomic nervous system comprises decreased markers of sympathetic activity, increased markers of parasympathetic activity, or both.

15. The method of any one of claims 1-14, wherein the bioceramic comprises kaolinite and tourmaline.

16. The method of claim 15, wherein the bioceramic comprises from about 20 wt % to about 80 wt % kaolinite and from about 1 wt % to about 30 wt % tourmaline.

17. The method of claim 15 or 16, wherein the bioceramic comprises at least one additional oxide.

18. The method of claim 17, wherein the one additional oxide comprises from about 1 to 20 wt % of the bioceramic.

19. The method of claim 17 or 18, wherein the one additional oxide is selected from the group consisting of aluminum oxide (Al₂O₃), silicon dioxide (SiO₂), titanium dioxide (TiO₂), magnesium oxide (MgO), and zirconium dioxide (ZrO₂).

20. The method of any one of claims 1-19, wherein the bioceramic composition comprises from about 40 wt % to about 60 wt % kaolinite (Al₂Si₂O₅(OH)₄) to about 5 wt % to about 15 wt % tourmaline to about 15 wt % to about 25 wt % aluminum oxide (Al₂O₃) to about 10 wt % to about 20 wt % silicon dioxide (SiO₂); and from about 1 wt % to about 20 wt % titanium dioxide (TiO₂); provided that the total amounts are by total weight of the composition.

21. The method of any one of claims 1-19, wherein the bioceramic composition comprises from about 40 wt % to about 60 wt % kaolinite (Al₂Si₂O₅(OH)₄) to about 5 wt % to about 15 wt % tourmaline to about 15 wt % to about 25 wt % aluminum oxide (Al₂O₃) to about 10 wt % to about 20 wt % silicon dioxide (SiO₂); and from about 1 wt % to about 20 wt % magnesium oxide (MgO); provided that the total amounts are by total weight of the composition.

22. The method of any one of claims 1-19, wherein the bioceramic composition comprises from about 40 wt % to about 60 wt % kaolinite (Al₂Si₂O₅(OH)₄) to about 5 wt % to about 15 wt % tourmaline to about 15 wt % to about 25 wt % aluminum oxide (Al₂O₃) to about 10 wt % to about 20 wt % silicon dioxide (SiO₂); and from about 1 wt % to about 20 wt % zirconium dioxide (ZrO₂); provided that the total amounts are by total weight of the composition.

23. The method of any one of claims 1-22, wherein the article is clothing.

24. The method of any one of claims 1-23, wherein the article is a shirt, a jacket, shorts or trousers.

25. The method of any one of claims 1-22, wherein the article is bedding.

26. The method of claim 25, wherein the bedding is selected from blankets, sheets, pillows, pillowcases, comforters, duvet covers, mattress covers, and mattress pads.

27. The method of claim 25 or 26, wherein the bedding is a blanket.

28. The method of any one of claims 1-27, wherein the article comprising the bioceramic is applied to the body of the subject for at least 1 hour, at least 2 hours, at least 3 hours, at least 4 hours, at least 5 hours, at least 6 hours, at least 7 hours, or at least 8 hours.

29. The method of any one of claims 1-28, wherein the article comprising the bioceramic is applied to the body of the subject daily, wherein the article comprising the bioceramic is applied to the body of the subject for at least 1 hour, at least 2 hours, at least 3 hours, at least 4 hours, at least 5 hours, at least 6 hours, at least 7 hours, or at least 8 hours each day.

30. The method of claim 29, wherein the article comprising the bioceramic is applied to the body of the subject daily over a period of time of at least 1 week, at least 2 weeks, at least 3 weeks, at least 4 weeks, at least 5 weeks, or at least 6 weeks.

31. The method of any one of claims 1-27, wherein the article comprising the bioceramic is applied to the body of the subject daily, wherein the article comprising the bioceramic is applied to the body of the subject for at most 1 hour, at most 2 hours, at most 3 hours, at most 4 hours, at most 5 hours, at most 6 hours, at most 7 hours, or at most 8 hours each day.

32. The method of claim 31, wherein the article comprising the bioceramic is applied to the body of the subject daily over a period of time of at most 1 week, at most 2 weeks, at most 3 weeks, at most 4 weeks, at most 5 weeks, or at most 6 weeks.
